(19) United States
(12) Patent Application Publication
(10) Pub. No.: US 2023/0112572 A1
(43) Pub. Date: Apr. 13, 2023

(54) Means and Method for Detecting Aberrant Erythroblasts

(71) Applicants: Stefan Schreier, Walderbach (DE); Premise Biosystems Co., Ltd., Samut Prakan (TH)

(72) Inventor: Stefan Schreier, Walderbach (DE)

(21) Appl. No.: 17/912,357

(22) PCT Filed: Mar. 19, 2021
(86) PCT No.: PCT/EP2021/057090

§ 371 Date: Sep. 16, 2022

(30) Foreign Application Priority Data
Mar. 19, 2020 (GB) 2003992.1

(51) Int. Cl. G01N 33/50; C12Q 1/68; G01N 33/68

(52) U.S. Cl. CPC G01N 33/5005; C12Q 1/68; G01N 33/6893; G01N 2800/10

(57) ABSTRACT

The present invention relates to a method for detecting, diagnosing, monitoring or prognosticating a pathologic status in a subject’s bone marrow including at least the step of determining the presence of aberrant erythroblasts in a subject’s bodily fluid sample. Also envisaged is the use of aberrant erythroblasts as a marker for diagnosing, detecting, monitoring or prognosticating a pathologic status in a subject’s bone marrow, as well as a composition for diagnosing, detecting, monitoring or prognosticating a pathologic status in a subject’s bone marrow, including means for the determination of the presence of (i) CD71 and/or GPA, (ii) CD45, (iii) nucleic acids of rare circulating cells and at least one of (iv) or (v): (iv) EpCam and (v) vimentin, in a subject’s sample.

### CROSS-REFERENCE TO RELATED APPLICATIONS

This application is the United States national phase of International Application No. PCT/EP2021/057090 filed Mar. 19, 2021, and claims priority to United Kingdom Patent Application No. 2003992.1 filed Mar. 19, 2020, the disclosures of which are hereby incorporated by reference in their entirety.

### BACKGROUND OF THE INVENTION

### Field of the Invention

The present invention relates to a method for detecting, diagnosing, monitoring or prognosticating a pathologic status in a subject’s bone marrow comprising at least the step of determining the presence of aberrant erythroblasts in a subject’s bodily fluid sample. Also envisaged is the use of aberrant erythroblasts as a marker for diagnosing, detecting, monitoring or prognosticating a pathologic status in a subject’s bone marrow, as well as a composition for diagnosing, detecting, monitoring or prognosticating a pathologic status in a subject’s bone marrow, comprising means for the determination of the presence of (i) CD71 and/or GPA, (ii) CD45, (iii) nucleic acids of rare circulating cells and at least one of (iv) or (v): (iv) EpCam and (v) vimentin, in a subject’s sample.

### Description of Related Art

The driving force of human tumorigenesis is multi-factorial and individually different often requiring years until systemic manifestation. This development presents itself from benign to malignant, dysplasia to neoplasia and local to systemic. Its complexity has prevented deep or complete understanding of cancer with respect to reversibility and speed of tumor growth. On a cellular level, the development of malignancy can be measured by the acquired number of stable genetic mutations per clone as well as the mutational and consequentially phenotypical heterogeneity between tumor cells. It is assumed that a cell must acquire at least 6 tumor relevant mutations until it is of malignant nature. Genetic alterations follow phylogeny, thus representing an evolutionary process and result in phenotypical changes, cell de-differentiation and consequently loss of function in an otherwise very specified cellular environment.

In many cases century-old grading systems are still in use, which are oriented on the degree of differentiation and translate into the assessment of tumor aggressiveness. The assessment of tumor aggressiveness, can be defined as the ability of the tumor to invade other tissue regions as well as to establish distant micro-metastasis at a given time period, may translate into prognostication and may have therapeutic implications. On a cellular level, tumor aggressiveness can be directly associated with clonal doubling time, grade of cell differentiation, as well as the variety of genetically distinct tumor cell populations. Therefore, aggressive tumors rapidly increase in mass, yet more importantly in clonal heterogeneity from pleomorphic neoplasms. This, thus, eventually raises the likelihood of some of the tumor cells to thrive in hostile or non-native distant tissue areas. Examples of particularly aggressive cancer types are adenocarcinomas of the pancreas.

The event of tumor cell spread and establishment of stable micro-metastasis marks the transition from a local to a systemic disease and consequently the evolution from a treatable to an often deadly disease. In particular, bone and bone marrow are assumed to constitute a supportive metastatic niche for several cancer types such as breast, prostate and lung tumors. Cancer patients with bone metastases are considered “high-risk” patients as little helpful means of treatment are available. Most metastatic development often remains undetected or unrecognized until latest phases of growth. It is commonly accepted that early detection of most cancer types is required to prevent early death by various treatment modalities and translates into the detection of disease before systemic manifestation. However, current diagnostic technology is typically limited in the recognition of early stage cancer, detecting early tumors either by chance or organ specific screening, such as is the case in breast cancer screening programs.

In liquid biopsy, the process of tumor cell shedding into the blood circulation is being investigated that is commonly accepted to support or represent the initiation of micro-metastasis. The factors contributing to a timely onset of tumor cell shedding at the individual and cancer type level is currently under investigation. Findings of bone marrow dwelling tumor cells, so called disseminated tumor cells at earliest stages in breast cancer patients supports the theory of early tumor cell dissemination and consequently proves the general ability to detect early stage cancer by cell-based liquid biopsy. It is further common knowledge that the egress of cells from bone marrow tissue into the circulation is elevated upon stress induction of any kind. Moreover, tissue lesions other than malignant tumors, such as myopathies, adenomas, colon polyps, pancreas cysts and many more have been identified to egress cells from affected areas into the blood circulation.

It is therefore hypothesized that any tissue lesion at any stage may cause cell egress into the circulation contributing to a circulating rare cell spectrum. This particular minute class of cell events, including inter alia malignant, benign, somatic and stem or progenitor cells amongst the vast majority of blood cells, is indicative, if not responsible for the development of a disease and therefore constitutes a noticeable diagnostic potential.

The bone-marrow is currently believed to be a major contributor to the rare cell spectrum. There is repair or maintenance motivated engraftment of bone marrow-derived cells of both the non-hematopoieitic and hematopoietic lineage upon pathological, but also physiological conditions. Furthermore, the bone marrow itself may be unbalanced, e.g. in inflamed bone marrow tissue, and consequently elicit uncontrolled cell egress into the circulation. Bone marrow cell egress needs thus to be discerned in a deliberate processes of the cellular immune as well as repair system and passive non-deliberate process caused by more or less severe damage of the bone marrow. Therefore, as the present inventors have found, system cancer evolution is detectable before systemic spread and may be detectable at its very beginning of in-situ lesions, hyperplasia or loci of inflammation.

The current diagnostic approach for a potential cancer patient is rather complex and intricate (Schünemann et al., Breast Cancer Screening and Diagnosis: A Synopsis of the European Breast Guidelines, Ann Intern Med, 2020, 172: 46-56; laccarino and Wiener, Diagnostic Evaluation After Lung Cancer Screening in Real-World Practice: More Questions Than Answers, 2020, Chest 157(2), 247-248). A screening positive patient must undergo means of confirming malignancy that is mostly restricted to investigations of tissue samples at the cellular level by a pathologist and involves exploratory tissue biopsy or cancer surgery. Separate investigations must follow as to assess the metastatic situation. Each test phase typically comes with limitations and drawbacks. A complete or correct picture of the cancer is not being given by each of the single diagnostic steps.

There is hence a need for an efficient and accurate diagnostic approach which allows to correctly and rapidly assess a tumor and its aggressiveness and to adapt potential therapy options.

### SUMMARY OF THE INVENTION

The present invention addresses this need and provides a method for detecting, diagnosing, monitoring or prognosticating a pathological status in a subject’s bone marrow comprising at least the step of determining the presence of aberrant erythroblasts in a subject’s bodily fluid sample. The invention is based on the finding that bone marrow-derived circulating rare cell populations constitute a reflection of the bone marrow condition. The present inventor has surprisingly found that determining the presence of aberrant erythroblasts in a subject’s bodily fluid sample and their molecular and phenotypical analysis can advantageously be used to complement and consequently improve diagnosis of cancer. The invention is accordingly based on the discovery of abnormal bone marrow native cells in the blood circulation of cancer and otherwise disease afflicted individuals. In the setting of known disorders that are associated with bone marrow damage, the finding of bone marrow-derived abnormal erythroblasts has to led the inventor to the premise that the spectrum of circulating erythroblasts is a reflection to certain extent of the bone marrow status in each individual. Consequently, this observation translates into the invention of analysis of the bone marrow without need of bone marrow aspiration, thus allowing to predict bone marrow damage with high reliability.

Hereby, the present invention may, in certain aspects, be specifically useful in the detection of bone metastasis. Furthermore, the present invention supports tumor staging with respect to systemic tumor cell spread by detecting tumor cell invasion of the bone marrow. Consequently, the present invention has two diagnostically implications for solid tissue cancer; one being a new powerful diagnostic window into tumor invasiveness and two, supporting delineation of metastasis. As such, the present invention relates to the potential replacement of bone marrow aspiration or claims equivalency to the diagnostic practice of bone marrow aspiration with respect to tissue cancer staging with all its advantages in patient care, sensitivity and costs. Furthermore, the present invention relates to simplified pre and post clinical diagnostic applications addressing acute conditions requiring immediate clinical response or monitoring over short periods.

In a preferred embodiment of the method as described above said sample is a venous blood sample or a peripheral blood sample, preferably an inferior vena cava sample or a portal vein sample.

In another preferred embodiment the determination comprises determining the ploidy of said erythroblasts and the potential presence of (i) a nuclear budding or lobulation phenotype, (ii) an internuclear bridge, (iii) two or more nuclei in a single cell, (iv) megaloblasts, (v) macronormoblasts, (vi) erythroblasts in synchronous cytoplasmic division, (vii) erythroblast aggregates comprising at least 3 adherent erythroblasts, (viii) increased ploidy within the erythroblasts’ nuclei.

In yet another preferred embodiment said aberrant erythroblast shows an increased ploidy, preferably has a bi- or multi-nuclear phenotype.

In yet another preferred embodiment said determination additionally comprises ascertaining the presence of at least CD71 and/or GPA, and optionally one or more of CD44, CD45, VAV1, Kell blood group protein and of nucleic acids in cells of the subject’s sample.

In a further preferred embodiment of the method according to the present invention, the identification of aberrant erythroblasts showing an increased intranuclear and/or intracellular ploidy, a nuclear budding or lobulation phenotype, an internuclear bridge, two or more nuclei in a single cell, a megaloblast appearance, a macronormoblast appearance and simultaneously the identification of a CD71⁺ (positive) and/or GPA⁺ (positive); and CD45⁻ (negative) biochemical status in said erythroblasts in the sample is indicative for bone marrow disorders such as myelodysplastic syndrome/acute myeloid leukemia, lymphomas, essential thrombocythemia or diabetes mellitus.

It is particularly preferred that said determination additionally comprises ascertaining the presence of EpCam and cytokeratin, and optionally of vimentin, in cells of the subject’s sample.

In a further preferred embodiment, the present invention relates to a method as defined herein above, wherein said determination additionally comprises ascertaining whether cells the subject’s sample are present in the form of cell clusters.

In a further preferred embodiment the identification of aberrant erythroblasts showing an increased intranuclear and intracellular ploidy, a nuclear budding or lobulation phenotype, an internuclear bridge, two or more nuclei in a single cell, a megaloblast appearance, a macronormoblast appearance and simultaneously the identification of a CD71⁺ (positive) and/or GPA⁺(positive); and CD45⁻ (negative) biochemical status in said erythroblasts in the sample, as well as the identification of EpCam⁺ (positive) or cytokeratin⁺(positive) cells in the sample is indicative for an invasive solid tissue cancer.

In yet another preferred embodiment said determination additionally comprises a morphological analysis of cells in said sample, preferably after May-Grünwald-Giemsa (MGG) staining of the cells.

It is particularly preferred that said morphological analysis of cells comprises a classification of the cells in the sample into:
class 1, wherein the sample comprises cells which are round or oval and which comprise one nucleus;
class 2, wherein the sample comprises cells which are round or oval and which comprise at least two nuclei;
class 3, wherein the sample comprises pairs of cells comprising a constriction; and
class 4, wherein the sample comprises aggregations of at least three round or oval cells with one nucleus or more nuclei.

In yet a further particularly preferred embodiment said class 1 comprises a further sub-classification of the cells into:
class 1a, wherein the sample comprises normal erythroblasts with a diameter of about 6.5 µm to about 12.4 µm with dense nucleus and high nucleus to cytoplasm ratio;
class 1b, wherein the sample comprises giant circulating erythroblasts of a diameter of about >12.5 µm, of at least one low density nucleus in diameter of about 6 to 10 µm and a low nucleus to cytoplasm ratio;
class 1c, wherein the sample comprises megaloblasts with nucleocytoplasmic asynchrony and moderate to high density chromatin and high nucleus to cytoplasm ratio; and
class 1d, wherein the sample comprises macronormoblasts with no nucleocytoplasmic asynchrony total condensation nuclei in a diameter of about 4 µm to 6 µm and a low nucleus to cytoplasm ratio.

In yet a further particularly preferred embodiment said class 2 comprises a further sub-classification of the cells into:
class 2a, wherein the cells are bi-nucleated;
class 2b, wherein the cells contain at least 3 nuclei.

In yet a further particularly preferred embodiment said class 3 comprises a further sub-classification of the cells into:
class 3a, wherein the cell shows no nuclear bridge; and
class 3b, wherein the cell shows a nuclear bridge and wherein the nuclei show an inequality in size, shape and/or chromatin density.

In yet another preferred embodiment said determination additionally comprises the determination of the ratio of at least one of: (i) class 1a cells vs. class 1c/1d cells; (ii) class 1a cells vs. class 2 cells; and (iii) class 1a cells vs. class 3 cells, and of clusters of circulating tumor cells (CTCs).

It is particularly preferred that the identification of at least one of (i) to (iii):
(i) an EpCam⁺ (positive) and CD45⁻(negative) circulating tumor cells (CTC) in the sample (marker 1);
(ii) a CTC cell cluster with a single cell diameter of 6 to 20 µm of CTCs showing marker 1 (marker 2);
(iii) an EpCam⁻ (negative) and vimentin⁺ (positive) CTC in the sample (marker 3); and additionally of
(iv) the presence of a ratio of at least one of (i) class 1a cells vs. class 1c/1d cells; (ii) class 1a cells vs. class 2 cells; and (iii) class 1a cells vs. class 3 cells of 50 or less to 0 is indicative for the malignancy of a solid tissue cancer.

In a further particularly preferred embodiment the identification of:
class 1a cells present in an amount of about 10 to 500 cells per ml in the sample and of class 1b/1c/1d cells present in an amount of about 1 to 10 cells per ml in the sample is indicative for a mild bone marrow damage; or
class 1a cells present in an amount about 1000 to 5000 cells per ml in the sample and of class 1b/1c/1d cells present in an amount of about 10 to 50 cells per ml in the sample is indicative for a moderate bone marrow damage; or
class 1a cells present in an amount greater than about 10 000 cells per ml in the sample and of class 1b/1c/1d cells present in an amount greater than about 10 cells per ml in the sample is indicative for a severe bone marrow damage.

In a further particularly preferred embodiment the identification of class 2a and class 2b cells in an amount of about 0.5 to 10 cells per ml of sample is indicative for a moderate bone marrow damage; or the identification of class 2a cells and class 2b in an amount of about 10 to 50 cells per ml of sample is indicative for a severe bone marrow damage

In yet another particularly preferred embodiment the identification of class 3a cells in an amount of about 1 to 10 cells per ml of sample is indicative for a moderate bone marrow damage; or the identification of class 3a cells are in an amount of about 10 to 50 cells per ml of sample and/or class 3b cells in an amount of about 1 to 10 cells per ml of sample is indicative for a severe bone marrow damage.

In yet another particularly preferred embodiment the identification of class 4 cells in an amount of a least one cell per ml of sample is indicative for a severe bone marrow damage.

In yet another particularly preferred embodiment the identification of a mild or moderate bone marrow damage and the identification of marker 1, 2 and/or 3 as defined above and the identification of a ratio of 50 or less to 0 as defined above is indicative for a cancer treatment related bone marrow damage of non-invasive cancer or a dormant cancer disease, minimal residual cancer disease or micro-metastasis in the bone marrow related to invasive solid tissue cancer.

In yet another particularly preferred embodiment the identification of a moderate bone marrow damage in the absence of class 2b cells per ml of sample and the identification of marker 1, 2 and/or 3 as defined above and the identification of a ratio of 50 or less to 0 as defined above is indicative for the presence of a progressive micro-metastasis in the bone marrow related to an invasive solid tissue cancer.

In yet another particularly preferred embodiment the identification of a severe bone marrow damage and the identification of marker 1, 2 and/or 3 as defined above and the identification of a ratio of 50 or less to 0 as defined above is indicative for the presence of an active micro-metastasis in the bone marrow related to an invasive solid tissue cancer.

In yet another particularly preferred embodiment, the identification of a severe bone marrow damage in the absence of class 2b cells per ml of sample and the identification of marker 1, 2 and/or 3 as defined above and the identification of a value of 50 or less to 0 as defined above is indicative for a bone metastasis related to invasive solid tissue cancer or primary bone cancer.

In a further aspect the present invention relates to the use of aberrant erythroblasts as a marker for diagnosing, detecting, monitoring or prognosticating a pathologic status in a subject’s bone marrow.

In a preferred embodiment, said aberrant erythroblasts are present in amounts as defined above.

In a further preferred embodiment said aberrant erythroblasts are used in combination with additional markers as defined above. It is particularly preferred that said pathologic status is malignant solid tissue cancer, more preferably non-invasive solid tissue cancer in the presence of mild or no bone marrow damage.

In yet another aspect the present invention relates to a composition for diagnosing, detecting, monitoring or prognosticating a pathologic status in a subject’s bone marrow, comprising means for the determination means for the determination of the presence of (i) CD71 and/or GPA, (ii) CD45, (iii) nucleic acids of rare circulating cells and at least one of (iv) or (v): (iv) EpCam and (v) vimentinin, a subject’s sample.

In a preferred embodiment, the means for the detection of EpCam are anti-EpCam antibodies MH99 and/or VU-1D9.

In a further preferred embodiment the composition additionally comprises means for the detection of the presence of one, two, three or all of CD44, CD24, CD133 and CD31.

### BRIEF DESCRIPTION OF THE DRAWINGS

FIG. 1 provides an overview of cell classes 1 to 4 according to the present invention.

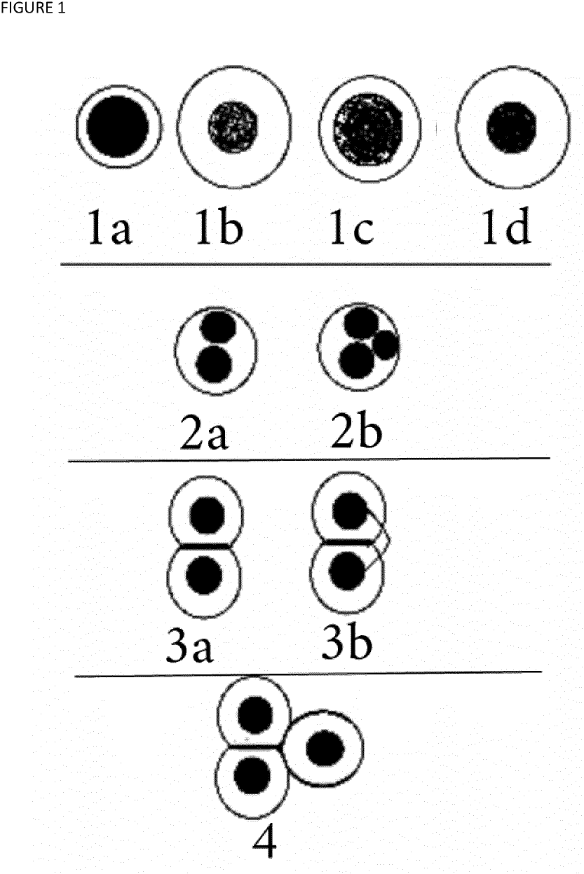

FIG. 2 shows a diagram of diagnostic procedure to ascertain invasiveness of a solid tissue tumor in bone marrow.

FIG. 3 shows a diagram of minimal residual disease testing.

FIG. 4 shows a mature erythroblast or normoblast, i.e. a class 1a cell according to the present invention.

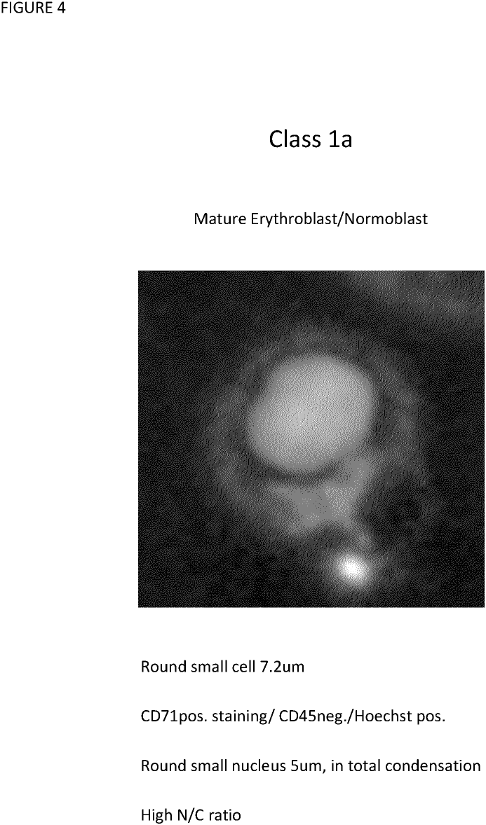

FIG. 5 shows an immature erythroblast, i.e. a class 1b cell according to the present invention.

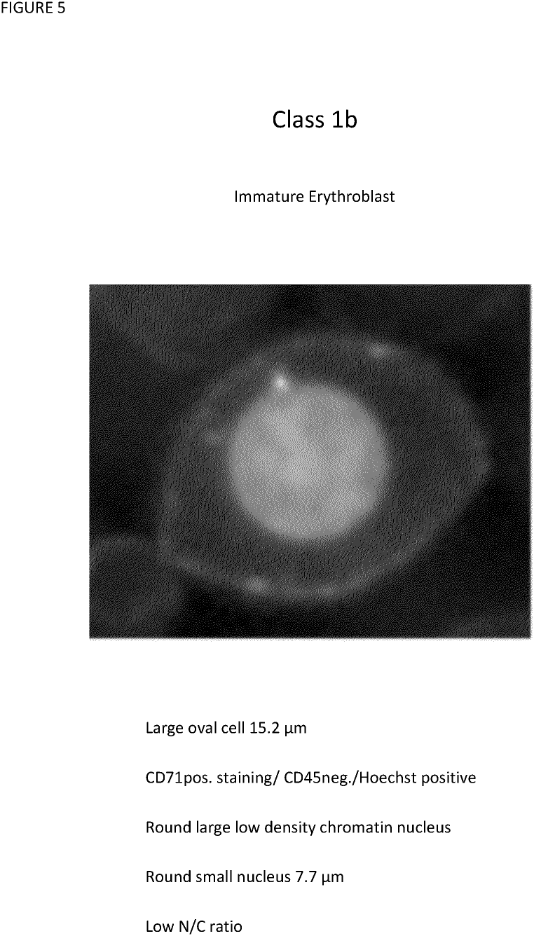

FIG. 6 shows a regular megaloblast, i.e. a class 1c cell according to the present invention.

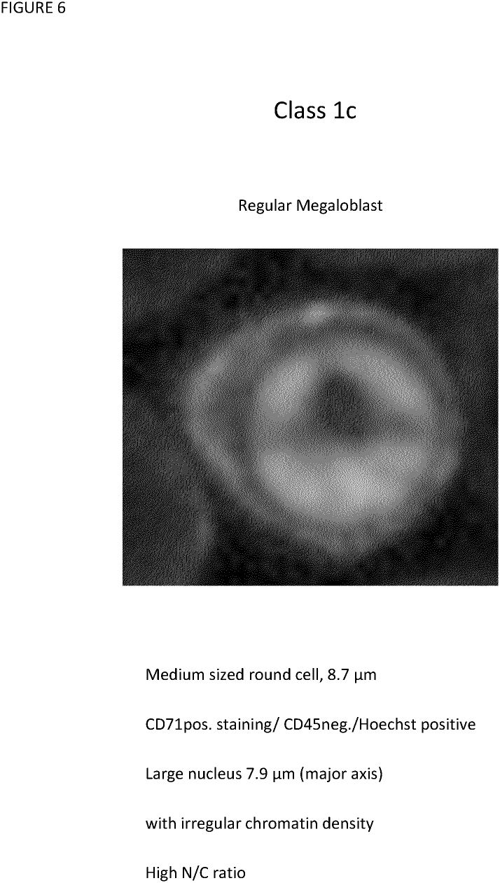

FIG. 7 shows a macronormoblast, i.e. a class 1d cell according to the present invention.

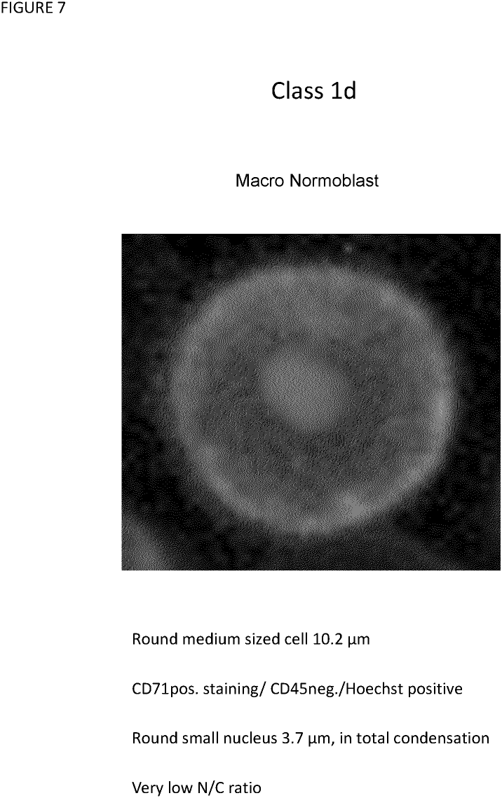

FIG. 8 shows a binucleated erythroblast, i.e. a class 2a cell according to the present invention.

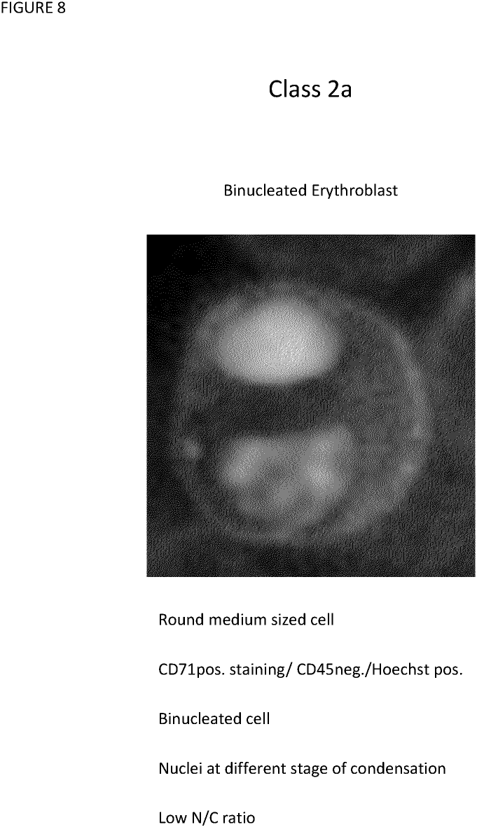

FIG. 9 shows a synchronously binucleated cell pair, i.e. a class 3a cell according to the present invention.

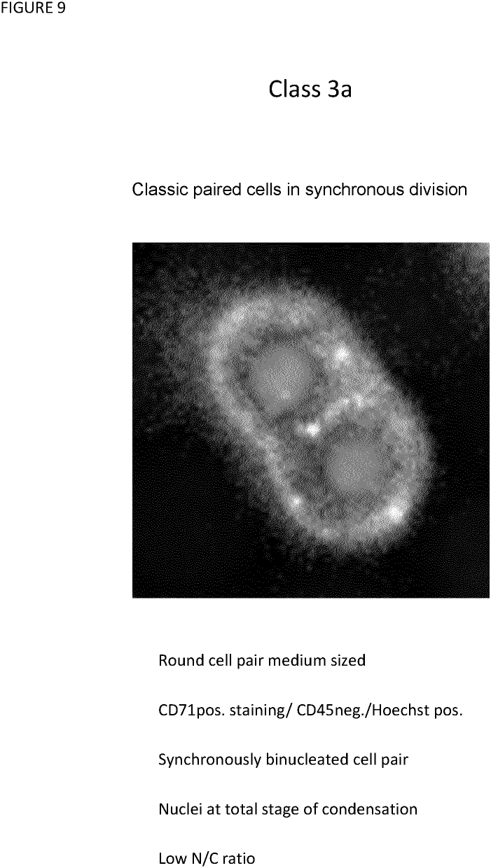

FIG. 10 depicts an aggregation of cells of different sizes, i.e. cells of class 4 according to the present invention.

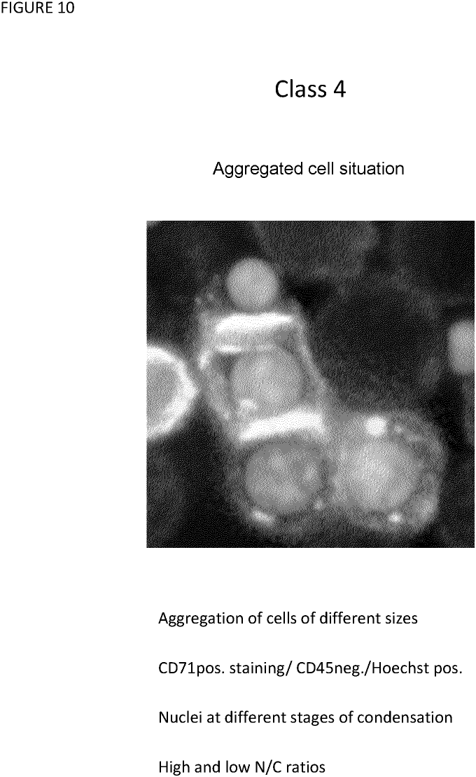

### DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise

In the context of the present invention, the terms “about” and “approximately” denote an interval of accuracy that a person skilled in the art will understand to still ensure the technical effect of the feature in question. The term typically indicates a deviation from the indicated numerical value of ±20 %, preferably ±15 %, more preferably ±10 %, and even more preferably ±5 %.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a method for detecting, diagnosing, monitoring or prognosticating a pathologic status in a subject’s bone marrow comprising at least the step of determining the presence of aberrant erythroblasts in a subject’s bodily fluid sample.

The term “bodily fluid sample” as used herein relates to any type of suitable sample of a bodily fluid. Typically, the sample is derived from a subject’s bodily fluid comprising one or more cells or cellular derivatives. Bodily fluids envisaged by the present invention may be include, for example, whole blood, fluids of the lymphatic system, saliva, nasal fluid, sputum, ear fluid, genital fluid, breast fluid, milk, colostrum, placental fluid, amniotic fluid, perspirate, synovial fluid, ascites fluid, cerebrospinal fluid, bile, gastric fluid, aqueous humor, vitreous humor, gastrointestinal fluid, exudate, transudate, pleural fluid, pericardial fluid, semen, upper airway fluid, peritoneal fluid, liquid stool, fluid harvested from a site of an immune response, fluid harvested from a pooled collection site, bronchial lavage, or urine. In further embodiments also material such as biopsy material, e.g. from all suitable organs such as lymph node material or bone marrow material, may be used. In order to be extracted, the biopsy material is typically homogenized and/or resuspended in a suitable buffer solution as described herein above. It is preferred that the sample is a venous blood sample or a peripheral blood sample. It is particularly preferred that the sample is an inferior vena cava sample, a pulmonary artery sample, a pulmonary vein sample or a portal vein sample. In specific embodiments, the sample is not a blood smear sample.

The collection of blood sample types comprising, for example, vena cava, pulmonary artery, pulmonary vein, portal vein excluding peripheral blood may have any suitable form or be performed in any suitable manner. Typically, the sample taking is invasive and may be best obtained in parallel to or shortly after surgery. Peripheral blood can preferably be collected from the cubital vein.

It is preferred that bodily fluid samples of all types, e.g. blood samples, are collected in volumes suitable for a subsequent processing, analysis, transport or storage. In particularly preferred embodiments, volumes from 3 ml to 30 ml, more preferably from 5 ml to 10 ml or 3 ml to 10 ml are used. In specific embodiments the bodily fluid samples are retained in a volume of 5 µl to 250 µl, e.g. after an enrichment step, or without enrichment. The cells may accordingly be retained in volume of 5 µl, 10 µl, 15 µl, 20 µl, 25 µl, 30 µl, 50 µl, 75 µl, 100 µl, 125 µl, 150 µl, 175 µl, 200 µl, 250 µl or any other volume in between the mentioned values. The term “enrichment” as used herein relates to any suitable form of enrichment procedure of cells, preferably an automated enrichment procedure. The enriched sample may, for example, be concentrated in 5 µl, 10 µl, 15 µl, 20 µl, 25 µl, 30 µl, 50 µl, 75 µl, 100 µl, 125 µl, 150 µl, 175 µl, 200 µl, 250 µl or any other volume in between the mentioned values of a suitable solution, e.g. a cell friendly solution such as phosphate buffered saline supplemented with 2%-10% fetal bovine serum or 0.5% bovine serum albumin. Further details can be derived from the Examples, e.g. Example 1, infra. It is further preferred that that the samples may be stored in common container tubes. Such container tubes may comprise additional ingredients which are required for stabilizing the sample, as pre-processing component, to avoid degradation or disintegration. In certain embodiments, in particular in the context of blood samples, the container tubes comprise EDTA such as EDTA-K2, EDTA-K3 or EDTA-2Na. Further preferred is the use of Na-Heparin. The mentioned ingredients and the samples may be stored at any suitable temperature, e.g. at room temperature. The storage may be performed for any suitable time. It is preferred that the storage is for not longer than 24 h. Further, it is preferred that the storage takes place in a place which is not light- or heat-exposed. It is particularly preferred that the storage takes place in the dark. The storage procedure may support cell viability of most cell types over a short period of time. In case of extended storage over a few days, e.g. 4 days, 5 day, 6 day, 7 days or more, preferably 7 days, the bodily fluid sample, e.g. the blood sample, needs to be subjected to a cell fixation or cell membrane stabilization reagent upon collection as known to the skilled person. A preferably envisaged method is commercially available under the name CellSave vacutainers (CellSearch®). In a further embodiment, prolonged storage may be supported by Cell-Free DNA BCT™, i.e. a blood collection device with a formaldehyde free stabilization reagent that preserves cell-free DNA in a blood sample for up to 14 days at RT.

It is preferred that the sample is a sample comprising aberrant erythroblasts in a low or very low quantity, e.g. as rare cells in a context of a high quantity of normal white blood cells. For example, the sample comprises aberrant erythroblasts in an amount of less than 3 x 10⁻⁶%, 3 x 10⁻⁵ %, 3 x 10⁻⁴ %, 0.01 % lower of the overall amount of white blood cells. This very low quantity of aberrant erythroblast reflects the etiology of the associated pathological status of the bone marrow, which is not caused by a mutation or genetic defect, which typically affects most or all erythroblasts, but is due to an indirect affect on the erythroblasts by interacting components such as pathogens or tumors.

The term “subject” or “patient” as used herein refers to a mammal, in particular to a human being. In specific embodiments, the subject is a human being in a post-pubertal age, e.g.an adult, e.g. of any age, or a person having passed through puberty, e.g. of any age. In a very specific embodiment, the subject is not a child or an adolescent in puberty or not a person who has not yet been in puberty. In further embodiments, the subject may be asymptomatic yet afflicted with a systemic pathology. Accordingly, the subject may, for example, show normal erythropoiesis upon onset of a disease which is negatively affected in due course of the disease. In a specific embodiment, the systemic component of a systemic pathology negatively affects erythropoiesis. It may, for example, be a pathogen or tumor cell that interacts, e.g. directly, with cells of the erythroid lineage in the red bone marrow. Further information may be derived from suitable literature sources such as Front Immunol., 2016; 7, 364. In another specific embodiment, the systemic component of a systemic pathology may negatively affect erythropoiesis. It may, for example, be a pathogen or tumor cell that is recognized by cells of the erythroid lineage. In another specific embodiment, the systemic component of a systemic pathology may negatively affect erythropoiesis such as a tumor cell or pathogen that generates inflammatory mediators in the red bone marrow. In another specific embodiment, the systemic component of a systemic pathology may negatively affect erythropoiesis such as a tumor cell, bone marrow cell or pathogen that changes the bone marrow micro-environment, e.g. by pH change, cell crowding, or reactive oxygen species deposition. In a further embodiment, the systemic component of a systemic pathology may negatively affect erythropoiesis comprising blood chemistry imbalances leading to tissue damage within the bone marrow.

The term “erythroblast” as used herein relates to a precursor of a red blood cells. Such a cell is typically subject to a cellular maturation process starting from a pro-erythroblast which is expected to exclusively take place in the bone marrow under physiological conditions. Cells in the latest stage of maturation of medullary erythroblasts are commonly referred to as normoblasts and can be found in the circulation of healthy donors at very low levels. Immature erythroblasts are less common (see Schreier et al., 2018, Annals of translational medicine, 6, 20). Normal or healthy erythroblast maturation typically comprises three morphological aspects, namely cell size, nucleus chromatin density, and nucleus/cell-ratio. Normal, non-aberrant erythroblasts are usually round cells containing one round nucleus in the centre throughout maturation and shrink as they mature starting from relatively large cells. These large cells may have a diameter of more than 20 µm. During maturation, the diameter typically decreases to about 7 µm or less. As the cell shrinks, the nucleus typically shrinks in conjugation with chromatin densification. This shrinkage typically takes place at different ratios in comparison to the cytoplasm. Therefore, upon shrinkage the N/C-ratio is maximized. Accordingly, normal or healthy erythroblasts may appear in different sizes and nucleus densities, but all show a characteristic high cell and nucleus roundness.

The term “aberrant erythroblast” as used herein relates to one or more phenotypical or molecular features which significantly differ from normal or healthy erythroblasts as described above. Typical erythroblast aberrations include mitotic processes found in the blood as opposed to erythroblast maturation, which is expected to occur under physiological conditions. Furthermore, erythroblast aberrations typically comprise synchronous and non-synchronized cell division. The non-synchronized cell division results in dysploidy (polyploidy).

A “circulating erythroblast” as used herein relates to an erythroblast, which is present in a bodily fluid, in particular blood or lymph fluid. A circulating erythroblast (cEB) may, for example, be a normal or non-aberrant circulating erythroblast. They are accordingly designated as class 1a or class 1b erythroblasts. A definition of these classes is provided herein below. Alternatively, the circulating erythroblast may have the form of an aberrant erythroblast as defined above. Circulating erythroblasts are typically bone marrow native.

The term “cellular abnormality” as used in the context of erythroblasts or erythroblast development herein relates to any asymptomatic or symptomatic, underlying or overt, chronic or acute, bone marrow cell intrinsic or external cell afflicted condition of the bone marrow that can be detected by circulating erythroblast quantification. The qualification of abnormality typically requires evaluation of counts above cut-off values for each circulating erythroblast class. Cellular abnormality is hence understood as a pathological status of the bone marrow, which can be detected via circulating erythroblasts.

According to the present invention the presence of non-aberrant circulating erythroblasts, i.e. of erythroblasts of class 1a or class 1b as defined herein, in a bodily fluid, e.g. blood or lymph fluid, which exceeds a certain threshold concentration, in particular as defined herein, indicates cellular abnormality. The mere presence of aberrant circulating erythroblasts in a bodily fluid, e.g. blood or lymph fluid indicates cellular abnormality. Their presence in the circulation accordingly provides valuable diagnostic information about the bone marrow condition when found in the bodily fluid, in particular blood.

Cellular aberrations in the bone marrow are known form the prior art, e.g. Goasguen et al., 2018, British journal of haematology 182.4, 526-533. However, there is so far no knowledge about the existence of particular aberrations associated with bodily fluids, e.g. the blood, of solid tissue cancer patients. In contrast to what is expected by experts in the field, the present inventors have found aberrant erythroblasts in a bodily fluid, in particular the blood, of solid tissue cancer patients. This finding strongly suggests a similar situation in the bone marrow. According to the present invention, detecting erythroblast abnormalities in a bodily fluid such as blood or lymph fluid may be used diagnostically for the detection of leukemia, anemias, diabetes or general bone marrow disorders. The detection of erythroblast abnormalities typically implicates the determination of a cell frequency given by cells per ml whole sample volume, e.g. whole blood as well as additional phenotypical and genotypical aberrations, e.g. polyploidy, of these cells.

Without wishing to be bound by theory, it is currently believed that circulating erythroblasts are a common find in healthy donors (Schreier et al., 2018, Annals of translational medicine, 6, 20; Fachin et al., 2017, Scientific reports 7, 1, 1-11). Circulating erythroblasts can be found at different stages of maturation, suggesting that the population of bone marrow derived rare cells and in particular circulating erythroblasts are a reflection of events in the bone marrow. The physiological circumstances of cell egress are currently believed to be either accidental or due to a deliberate process. Accidental egress of mature erythroblasts could be explained by their proximity to the circulation in the peri-vascular niche. Herein, the clonal or mitotic situation of bone marrow dwelling erythroblasts may also cause localized competition for space within erythroblastic islands and ultimately leading to cell egress at the blood barrier in the bone marrow. However, this does not explain the finding of immature erythroblasts usually being part of the endosteal niche, suggesting a more active role, as such deliberate process of cell egress. It is assumed that circulating erythroblasts egress is potentiated under pathological conditions. With wishing to be bound by theory, it is expected that pathological conditions within the bone marrow are also reflected by the situation in the circulation. Thus, according to the current invention, an erythroblast signal found, for example, in the blood alludes to conditions or imbalances in the bone marrow.

A specific type of imbalance can, for example, be caused by disseminated tumor cells (DTC) that have settled in the peri-vascular niche or deeper within causing uncontrolled or unaccounted disturbances in the bone marrow micro-environment, such as localized inflammation. Bone and bone marrow nesting by disseminated tumor cells may be associated with specific expression of adhesive membrane molecules that bind them to marrow stromal cells and bone matrix, so that bone marrow becomes a vital “breeding ground” for disseminated tumor cells. It is further believed that bone marrow is a “training ground” for metastatic unfit circulating tumor cells (CTCs). Accordingly, rather than being a site of metastasis itself, it constitutes an essential diagnostic target for the purpose of delineation of metastatic processes. Consequences of nesting may, for example, be impairment of the peripheral bone marrow blood barrier function and bone-marrow cell hyperplasia. Both events may then lead to non-deliberate egress of normal and aberrant bone marrow cells as well as tumor cells, then being detectable in sum as circulating rare cells in the peripheral blood circulation.

Within the context of the present invention, it is assumed that bone marrow derived circulating erythroblasts are taking vital part in the delineation of metastasis and thus allow the identification of early developmental stages or processes which take place before detectable secondary tumor growth. A typical scenario, which is envisaged by the present invention, is the presence of micro-metastases that - when overlooked - cause incorrect staging and significantly increase the risk of inefficient treatment. It is expected that a micro-metastasis is a consequence of, or follows from, or coincides with bone marrow infiltration.

In view of research about genetic instability, delineation is believed to stretch back not only to micro-metastasis or pre-malignancy but also to pre-cancerous lesions. In line with the teaching of the present invention, the identification and characterization of tumors should follow different analytical paths, preferably prior to therapy, including genetic analysis of certain driver mutations being most indicative for the potential to invade and thrive at distant sites in the body. In contrast to the approach of predicting invasiveness, the currently envisaged diagnostic effort of metastasis delineation is part of the characterization of the individual tumor evolution and relies on the assessment of the invasion status at certain time points and locations. The most critical location is the site of the bone marrow.

Tumor cell dissemination is believed to be an early event, most likely as early as pre-malignancy. In consequence, tumors must shed viable tumor cells into the circulation at earliest time points giving rise to circulating tumor cells (CTCs). Importantly, detecting circulating tumor cells may not allow a diagnostic statement on tumor invasiveness, but only allows a diagnostic statement on presence of a neoplastic lesion. Sisseminated tumor cells, on the other hand, are a clear prove of invasiveness for being found and settled at sites other than the primary tumor. As taught by the present invention disseminated tumor cell identification by cell-based liquid biopsy in the peripheral blood system requires indirect evidence for bone marrow infiltration. It can accordingly be achieved via the identification of different classes of circulating rare cells that can be unambiguously identified as being bone marrow-derived. These cells, i.e. the circulating tumor cells, are, according to the teaching of the present invention, indicative for bone marrow imbalances.

The term “pathologic status in a subject’s bone marrow” as used herein relates to a disease-state or pre-disease-state in the bone marrow tissue of a subject. Such a disease- or pre-disease-state may, for example, be a bone marrow associated disorder which leads to leukemia and anemia, or it may be or be induced by a bone marrow damage, which can be, for example, a mild, moderate or severe bone marrow damage. The disease- or pre-disease state may further be associated to an existing or developing tumor or neoplasm, or preforms thereof. In a very specific embodiment the pathologic status in a subject’s bone marrow is not associated with a disease described for a non--human animal, in particular for a mouse. In further very specific embodiments, the pathologic status may be a disease-state or pre-disease-state in the bone marrow tissue of a subject other than a congenital dyserythropoietic anemia in a subject in a pre pubertal or pubertal age. In further very specific embodiments the pathologic status may be a disease-state or pre-disease-state in the bone marrow tissue of a subject other than mutations in the genes KLF1, CDAN1, SEC23B or GATA-1, e.g. a E325K mutation in the KLF1 gene and/or caused by said mutation. Further information may be derived from suitable literature sources such as Haematologica, 2012; 97(12): 1786-1794. In specific embodiments, the pathologic status in a subject’s bone marrow may be malignant solid tissue cancer. Examples of such malignant solid tissue cancers include carcinomas in the lung, breast, and colon. Alternatively, it may be a tumor or pre-tumor form in the form of a metastasis or tumor derived cell aggregate. In further embodiments, the disease may be a myelodysplastic syndrome, an acute or chronic myeloid leukemia, an acute or chronic lymphocytic leukemia, a lymphoma involving the bone marrow, an essential thrombocythemia or diabetes mellitus, or a non-neoplastic acquired anemia including an iron deficiency, arsenic toxicity, and vitamin B12 deficiency caused anemia.

In specific embodiments, the pathologic status in a subject’s bone marrow is a bone marrow injury caused by metastases, or is a bone marrow injury caused by an invasive tumor.

It is particularly preferred that the pathological status in a subject’s bone marrow is not due to a genetically caused erythrocyte or erythroblast aberration, e. g. due to a gene defect or a mutation in an erythrocyte, an erythroblast or a hematopoiesis-associated cell or corresponding stem cell, e.g. one of the gene defects mentioned above. It is further preferred that the pathological status in a subject’s bone marrow is connected to a systemic pathology, in particular a systemic component of a systemic pathology which negatively effects erythropoiesis such as a pathogen or a tumor cell.

One important aspect of a pathologic status in a subject’s bone marrow is bone marrow damage. Typically, bone marrow damage is caused by disorders of the bone marrow such as leukemia or anemia. Bone marrow damage may, further, be encountered upon systemic inflammation, which can include diseases such as diabetes (see Fadini et al., 2014, 3, 8, 949-957), carcinomas and lymphomas or acquired by cancer therapy with respect to radiation induced or cytotoxic drug induced bone marrow injury.

A “mild bone marrow damage” as described herein refers to a clinical situation in which the subject is feeling asymptomatic and healthy (subjective feeling) and may nor may not show abnormal blood results as common the art. This category may include static situations as well as dynamic developments with respect to lifestyle or manifested pathologies, respectively. With respect to diseases, mild damage typically indicates the onset or early stages of disease development. For example, a mild bone marrow damage may be associated with solid tissue cancers, wherein mild damage may represent tumor cell dormancy or minimal residual disease as present in the bone marrow with either way of development; damage reversion or progression. With respect to lifestyle, mild damage is typically immanently reversible upon lifestyle change. A mild bone marrow damage may lead to a physiological egress of erythroblasts, which may be similar to the egress of other cells derived from stem or progenitor cells, which are bone marrow based. The egress of erythroblasts even in higher numbers may not necessarily be associated with physical damage of the vascular niche, thus suggesting physiological normality. Therefore, cellular abnormality with respect to mild bone marrow damage is, according to the teaching of the present application, associated with dysregulated cell maturation.

A “moderate bone marrow damage” as described herein relates to an already pathological status, e.g. at an earlier stage of a disease that indicates or predicts an increase in severity, i.e. will lead to a severe bone marrow damage, or predicts continued disease evolution, i.e. local to systemic disease. It is equivalent to a medium bone marrow damage. It may also predict the onset of secondary disease complications such as diabetic neuropathy. Furthermore, erythroblast mitosis, which is a dysregulation of cellular processes and, for example, indicative for a chemical injury such as inflammation processes, may be associated with a moderate bone marrow damage. These processes further may or may not coincide with a physical destruction of the bone marrow and bone matrix as described below. A rating as “moderate bone marrow damage” typically depends on the quantities of identified aberrant erythroblasts.

A “severe bone marrow damage” as described herein indicates a pathological status of individuals usually at a late stage condition. In the context of cancer, this category holds the likelihood of bone metastasis. In line with the teaching of the present application, it is assumed that bone tumors are typically initiated by one or more disseminated tumor cells in the bone marrow, in particular hematopoietic niche dwelling tumor cells. These cells can dysregulate osteoblastic and osteoclastic cell maturation leading to osteosclerosis and osteolysis, respectively in an interplay of bone matrix destruction and tumor cell growth. A physical damage of the micro-vascularity within the bone marrow may accordingly lead to the presence of particular large aggregates of circulating erythroblasts, i.e. of class 4 erythroblasts as defined herein. Furthermore, involved cytokines were shown to have an effect on the erythroblast maturation leading to mitotic cell findings within the circulation, i.e. the identification of class 2 and 3 erythroblasts as defined herein, in a bodily fluid, in particular blood. The presence of class 4 erythroblasts in a bodily fluid, e.g. blood, is thus assumed to be a consequence of a physical destruction in the bone marrow, hence indicating a severe injury of the bone marrow.

Bone marrow damage as described herein leads to and/or may be detected via the presence of cellular abnormality, in particular erythroblast aberration, in particular in the context of leukemias and anemias.

A further example of an association of erythroblast aberration and a disease is diabetes and the presence of solid tissue cancer. Without whishing to be bound by theory, it is assumed that several genetic or epi-genetic mutations may be the cause of one typical phenotypical appearance.

Another example is myelodysplastic syndrome with erythroid dysplasia, which typically includes nuclear changes like abnormal chromatin clumping, bi/multinuclearity or nuclear bridges. In the context of this syndrome significant defects in histone release of EBs may be found as a result of defective caspase 3 processes which are assumed to lead to the inhibition of chromatin condensation, and terminal differentiation required for EB maturation then contributing to the pathogenesis of megaloblastoid changes in dyserythropoiesis (see Baobing et al., 2019, Cancer medicine, 8.3, 1169-117). In a further example, a genetic factor has been identified for CDAII, a specific mutation in the SEC23B gene, which was ascribed to causing multinucleation (Pellegrin et al., 2019 British journal of haematology, 184.5, 876).

The term “detecting a pathologic status in a subject’s bone marrow” as used herein means that the presence of a pathologic status in a subject’s bone marrow as defined herein above, preferably a bone marrow associated disorder associated to leukemia or anemia, or a bone marrow damage, or a developing or existent tumor or neoplasm such as a malignant solid tissue cancer, in a subject may be determined or that such a disease or disorder may be identified in the subject.

The determination or identification of a pathologic status in a subject’s bone marrow as defined herein above may be accomplished by a comparison of phenotypical or genotypical markers associated with said pathologic status of the present invention and a normal or healthy control.

A pathologic status in a subject’s bone marrow as defined herein above may be detected when at least one, preferably more than one phenotypical or genotypical marker is present or significantly increased in comparison to a normal/healthy control situation or level as defined herein. In a preferred embodiment this control situation or level as defined herein is obtained from or representative for a healthy subject that is not afflicted with a pathologic status its bone marrow as defined herein.

In another preferred embodiment of the present invention a pathologic status in a subject’s bone marrow may be detected if the presence of level of the phenotypical or genotypical marker is similar to a control situation or level as defined herein, e.g. derived from a bone marrow of a subject which has independently been diagnosed to have a pathologic status in its bone marrow.

In preferred embodiments, the detection of a pathologic status in a subject’s bone marrow is linked to the detection of aberrant erythroblasts in a subject’s bodily fluid, preferably a blood sample.

The term “diagnosing a pathologic status in a subject’s bone marrow” as used herein means that a subject may be considered to be suffering from a disorder or disease leading to the pathologic status as defined herein. Such disorder may include a bone marrow associated disorder associated to leukemia or anemia, or a tumor or neoplasm such as a malignant solid tissue cancer.

The diagnosis of a pathologic status in a subject’s bone marrow as defined herein above may be accomplished by a comparison of phenotypical or genotypical markers associated with said pathologic status of the present invention and a normal or healthy control. In specific embodiments, the diagnosis may comprise additional steps, e.g. independent histologic, biochemical or genetic examinations. Further envisaged is an AI-based comparison approach on the basis of available training data from healthy and independently diagnosed pathologic subjects.

A pathologic status in a subject’s bone marrow as defined herein above may be diagnosed when at least one, preferably more than one phenotypical or genotypical marker is present or significantly increased in comparison to a normal/healthy control situation or level as defined herein. In a preferred embodiment this control situation or level as defined herein is obtained from or representative for a healthy subject that is not considered to be suffering from a disorder or disease leading to the pathologic status as defined herein.

The term “diagnosing” also refers to the conclusion reached through the above comparison process, in particular specific processes described herein below.

The term “monitoring a pathologic status in a subject’s bone marrow” as used herein relates to the accompaniment of a diagnosed or detected pathologic status in a subject’s bone marrow, e.g. during a treatment procedure or during a certain period of time, typically during 1 month, 2 months, 3 months, 4 months, 5 months, 6 months, 1 year, 2 years, 3 years, 5 years, 10 years, or any other period of time. The term “accompaniment” means that states of disease as defined herein and, in particular, changes of these states of disease may be detected by comparing the phenotypical or genotypical markers associated with said pathologic status according to the present invention in a sample to a normal or healthy control as defined herein. In another embodiment an established, e.g. independently established, pathologic bone marrow, or a cell line derived therefrom can be used as a positive control. Such an accompaniment may be performed in any type of periodical time segment, e.g. every week, every 2 weeks, every month, every 2, 3, 4, 5, 6, 7, 8, 9, 10, 11 or 12 month, every 1.5 year, every 2, 3, 4, 5, 6, 7, 8,9 or 10 years, during any period of time, e.g. during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively.

The term “prognosticating a pathologic status in a subject’s bone marrow” as used herein refers to the prediction of the course or outcome of a diagnosed or detected bone marrow associated disorder associated to leukemia or anemia, or a bone marrow damage, or a developing or existent tumor or neoplasm such as a malignant solid tissue cancer as defined herein during a certain period of time, during a treatment or after a treatment. The term also refers to a determination of chance of survival or recovery from the disease, as well as to a prediction of the expected survival time of a subject. A prognosis may, specifically, involve establishing the likelihood for survival of a subject during a period of time into the future, such as 6 months, 1 year, 2 years, 3 years, 5 years, 10 years or any other period of time.

The determination of the presence of aberrant erythroblasts in a subject’s bodily fluid, e.g. blood, may comprise any suitable activity or step known to the skilled person or derivable from suitable literature sources such as Fachin et al., 2017, Scientific reports, 7.1, 1-11 and Schreier et al., 2018, Annals of translational medicine, 6.20. In preferred embodiments, the determination comprises at least determining the ploidy of the erythroblasts. As known to the skilled person, the term “ploidy” refers to the number of complete sets of chromosomes in a cell. Mammal cells typically have a diploid level, i.e. they have 2 sets of chromosomes. In the case of normal or healthy erythroblasts the ploidy level is also diploid. In aberrant erythroblasts, the ploidy level may be different from 2, e.g. > 2 such as 4, 6 or higher. The ploidy level may be reflected within a single nucleus (with more chromosomes), or in more than one nucleus per cell, or in both, an increased ploidy within a nucleus and more than one nucleus per cell.

In a very specific embodiment the determination of the presence of aberrant erythroblasts is performed exclusively in a subject’s bodily fluid. Accordingly, in this very specific embodiment the determination of the presence of aberrant erythroblasts in other tissues such as the bone marrow is excluded. In another very specific embodiment, the combination of analysis steps as disclosed in Jaffray et al., 2013, Blood Cells, Molecules and Diseases, 51, 71-75 is excluded.

Methods to determine ploidy in an erythroblast would be known to the skilled person or can be derived from suitable literature sources such as Zhang et al., 2020, Cancer Letters, 469, 355-366. It is preferred to determine the ploidy of erythroblasts in a bodily fluid sample with fluorescent detection, preferably with fluorescent in situ hybridization technology, e.g. as described in FISH. Further details may be derived from suitable literature sources such as Ehtisham et al., 2016, International Research Journal of Clinical Medicine, 1, 4, 23-29.

Determining the ploidy of erythroblasts in the bodily fluid may additionally comprise the determination of the presence of one or more phenotypic states of an erythroblast. These phenotypes can be detected or seen, when analyzing the erythroblasts after nuclear staining and subsequent microscopy. Preferred staining approaches use dyes such as NucSpot, RedDot, DMAO, SYBR Green, Thiazole Green, Thiazole Orange, DAPI, acridine orange, p-phenylenediamine, DRAQ5, Ethidium Homodimers I or III, or Hoechst33342. The microscopy is preferably a UV microscopy or UV-VIS microscope. Further preferred for the determination of the ploidy of erythroblasts are automated imaging systems, which allow automated image analysis, e.g. as defined herein.

Among these phenotypic states are a nuclear budding or lobulation phenotype. In this phenotype, the erythroblast shows within the nucleus a protuberance or excrescence. The nucleus is typically present in a condensed form.

Another example of a phenotypic state of an aberrant erythroblast is the presence of an internuclear bridge. In this phenotype the cell comprises at least two nuclei, which are linked by a DNA comprising structure having the form of a bridge, which is also referred to as chromatin bridge. It is preferred to perform the determination at a 400 x magnification, or a higher magnification, or at a resolution of about 6.7 µm per pixel or less. Further details can be derived from suitable literature sources such as Bethlenfalvay et al., 1986, American journal of hematology, 21.3, 315-322.

A further example of a phenotypic state of an aberrant erythroblast is the presence two or more nuclei in a single cell. A single cell may, for example, comprise 2, 3, 4, 5 or more nuclei. These nuclei may be completely separated or may appear in division similar mitotic processes during ana- and telophase. Two or more may be connected via an internuclear bridge as mentioned above.

Another example of a phenotypic state of an aberrant erythroblast is the occurrence of a megaloblast. The term “megaloblast” as used herein relates to an erythroblast, which has a large nucleus in relation to the size of the erythroblast. A megaloblast is typically 50 to 150% or 1.5 x to 2.5 x larger than a normoblast. It is assumed that megaloblasts occur due an impairment in DNA replication that delays nuclear maturation and cell division. Megaloblasts typically show a dissociation o between nuclear and cytoplasmic maturation.

A further encountered example of a phenotypic state of an aberrant erythroblast according to the present invention is the occurrence of macronormoblast. The term “macronormoblast” as used herein relates to an unusually large erythroblast, which may be as large as a megaloblast, or larger than a megaloblast. It typically contains very small nuclei displaying total chromatin condensation. Furthermore, the cell maturation of this cell type is asynchronous and dissociated. In contrast to a megaloblast, a macronormoblast does not show a dissociation between nuclear and cytoplasmic maturation.

Another example of a phenotypic state of an aberrant erythroblast according to the present invention are erythroblasts in synchronous cytoplasmic division. These erythroblasts are groups of 3 erythroblasts which are in the same phase of cytoplasmic division. Erythroblasts in synchronous cytoplasmic division are typically class 3 cells as defined herein, which may be either with or without bridging). They consist of two pairs of cells with either equal or unequal size with respect to cell diameter and nucleus area. The pairs may be clearly separated or appearing as one entity suggesting different states of cytokinesis. The nuclei are always locally separate suggesting finalization of telophase.

Further envisaged is an example of a phenotypic state of an aberrant erythroblast, wherein erythroblast aggregates occur. These aggregates typically comprise at least 3, e.g. 3, 4, 5, 6, 7, 9, 10 or more adherent erythroblasts.

A further example of a phenotypic state of an aberrant erythroblast an increased ploidy within the erythroblasts’ nuclei. Accordingly, the nucleus of an erythroblast may comprise 4, 6, 8 or more sets of chromosomes, i.e. have a ploidy level of 4, 6, 8 or more. In specific embodiments, e.g. in cases associated with leukemia, the erythroblasts may also comprise 3, 5, 7, 9 or more sets of chromosomes, i.e. have a ploidy level of 3, 5, 7, 9 or more. Nuclei of such erythroblasts may have an enlarged size and/or a higher density.

In preferred embodiments of the present invention, the aberrant erythroblast shows an increased ploidy, e.g. a ploidy level of 4, 6, 8 or more. In particularly preferred embodiments, the aberrant erythroblast shows a bi- or multi-nuclear phenotype. A “bi-nuclear phenotype” as used herein means the presence of two nuclei per cell. These nuclei may be separated or be linked by an internuclear bridge as described herein above. A “multi-nuclear phenotype” refers to the presence of more than 2 nuclei per cell, e.g. 3, 4, 5, 6, 7, 8 or more. These nuclei may be separated or (at least some) may be linked via internuclear bridges as described above.

In a further embodiment, an acidified serum test is performed with a bodily fluid sample, in particular a blood sample, in order to detect, diagnose, monitor or prognosticate a pathologic status in a subject’s bone marrow. This test, which is also known as Ham test, involves placing red blood cells in mild acid. This test essentially checks whether red blood cells become more fragile when they are placed in mild acid. The test is typically based on visual observation and allows for a distinction of a positive or negative result.

In case an acidified serum test as described above shows red blood cell lysis in a blood sample and if aberrant erythroblasts showing an increased ploidy as described above are identified in said sample, it can be deduced that the subject the sample is derived from is afflicted by a pathologic status in a its bone marrow. Specifically, such a subject is assumed to be afflicted by the bone marrow associated disease of hereditary erythroblastic multinuclearity (congenital dyserythropoietic anemia type II).

In a further set of embodiments, the present invention envisages the determination of one or more biochemical or molecular markers associated with erythroblasts. Accordingly, the biochemical status of one or more of these markers may be determined. The term “biochemical status” as used herein relates to the presence of absence of markers, e.g. proteins or derived forms thereof, and/or to the amount or concentration of said proteins. For the biochemical status “positive” is required to detect a protein with suitable, preferably standardized, methods known to the skilled person above a threshold level known to the skilled person, e.g. derivable from suitable literature or internet sources. More preferably, corresponding information as well as calibration tests etc. may be provided by manufacturers offering a kit for the detection of the corresponding biomarker. The skilled person would be able to adjust the conclusion on the presence of a biomarker to the specific parameters of the test employed.

Analogously, for the biochemical status “negative” it is required to detect no protein with suitable, preferably standardized, methods known to the skilled person or to detect a protein below a threshold level known to the skilled person, e.g. derivable from suitable literature or internet sources. More preferably, corresponding information as well as calibration tests etc. may be provided by manufacturers offering a kit for the detection of the corresponding biomarker. For example, a so called isotype control test may be performed. Such a test is typically required in fluorescence flow cytometry and allows to test the level of non-specific binding of antibody isotypes. This level is usually interpreted as background noise level. The skilled person would be able to adjust the conclusion on the absence of a biomarker to the specific parameters of the test employed, or to background noise level as defined above.

It is, in certain embodiments, envisaged to determine, in order to detect, diagnose, monitor or prognosticate a pathologic status in a subject’s bone marrow, the presence of the marker CD71 on a cell, in particular on an erythroblast.

CD71, which is also known as transferrin receptor protein 1 (TfR1) is assumed to be required for iron import form transferrin into cells by endocytosis. The protein is a transmembrane glycoprotein composed of two disulphide-linked monomers. It is considered as erythroid precursor marker. Without wishing to be bound by theory, it is hypothesized that CD71 is selectively and ubiquitously expressed at high levels in erythroid precursors of all maturation stages, including normal and dyspoietic bone marrows (see Dong et al., 2011. Am J Surg Path, 35(5), 723-732). Further information can be derived from Schreier et al., 2018, Annals of translational medicine, 6.20). For the detection of CD71 any suitable antibody or other binding entity may be used. It is preferred to employ monoclonal antibodies. Examples of such antibodies include OKT9 and H68.4.

It is, in certain embodiments, envisaged to determine, in order to detect, diagnose, monitor or prognosticate a pathologic status in a subject’s bone marrow, the presence of the marker GPA on a cell, in particular on an erythroblast. GPA may be determined together with CD71, or in other embodiments, alternatively to CD71.

GPA, which is also known as glycophorin A or CD235ais a 151 amino acid sialoglycopro-tein, which is expressed exclusively on the cell membrane of the erythroid cell line from early stage till the formation of mature red blood cells at about 500,000 copies per cell. The gene for glycophorin resides on chromosome 4 and has 2 allelic forms: M and N, which differ in two amino acids. The M group possesses Ser1 and Gly5 while the N group has Leu1 and Glu5. This marker does not distinguish between blast cells and matured cells of the erythroid cell line and therefore, is used as auxiliary marker to confirm the nature of the positively identified cells in question (see also Grant et al., 1974, Proceedings of the National Academy of Sciences, 71.12, 4653-4657). CD235a is usually detected by monoclonal antibody ligand. The detection antibodies can see both the M and N alleles. In applications tested, relevant clones such as 10F7MN are pre-titrated and tested by flow cytometric analysis of normal human blood cells. This can be used at 5 µL (0.25 µg) per test. A test is defined as the amount (µg) of antibody that will stain a cell sample in a final volume of 100 µL. Cell number should be determined empirically but can range from 10⁵ to 10⁸ cells/test. In the said method, flow cytometry is unfit for detecting rare events, hence requires analysis by microscopy.

In further embodiments, the presence of additional biochemical or molecular markers on a cell, in particular on an erythroblast, is envisaged. These additional markers comprise CD44, CD45, CD24, CD133, CD31, VAV1, Kell blood group protein. These markers may be determined individually together with CD71 and/or GPA, e.g. as combination of CD71 and/or GPA plus CD44, CD71 and/or GPA plus CD45, CD71 and/or GPA plus VAV1 etc., or they may be determined as differential group in combination with CD71 and/or GPA, e.g. CD71 and/or GPA plus CD44 and CD45 and VAV1; or CD71 and/or GPA plus CD45 and VAV1 and Kell blood group protein; or CD71 and/or GPA plus CD44 and CD45 and VAV1; or CD71 and/or GPA plus CD44 and CD45 and VAV1 and Kell blood group protein etc. The group may comprise any 2, 3, or 4 items of the marker group CD44, CD45, CD24, CD133, CD31, VAV1, Kell blood group protein.

CD44 is a receptor for hyaluronic acid and can interact with different ligands such as oestopontin, collagens or MMPs. It is a cell-surface glycoprotein involved in cel-cell interactions, cell adhesion and migration. The protein exists in several isoforms due to the presence of splice variants. It may further comprise different sugar residues. One variety, a sialofucosylated version, which is known as HCELL glycoform is assumed to serve as ligand for lectins. Preferably, the form phagocytic glycoprotein 1 of CD44 is used as biochemical marker, e.g. via a suitable monoclonal antibody. CD44 expression is assumed to be maturation state dependent, suggesting high expression during early and low expression during late stages. Further information may be derived from suitable literature sources such as Chen et al., 2009, Proceedings of the National Academy of Sciences 106.41, 17413-17418. For the detection of CD44 any suitable antibody or other binding entity may be used. It is preferred to employ monoclonal antibodies. Examples of such antibodies include IM7 and Hermes-1.

CD45 is protein tyrosine phosphatase, receptor type, i.e. a transmembrane protein that is present in several isoforms on all differentiated hematopoietic cells, except erythrocytes and plasma cells. CD45 contains an extracellular domain, a single transmembrane segment, and two tandem intracytoplasmic catalytic domains, and thus belongs to the receptor type PTP family. It is a regulator of T- and B-cell antigens receptor signaling. The current invention envisages the recognition of at least five isoforms of the CD45antigen as currently known, i.e. RA, RO, RB, RAB, RBC and RABC. It preferred to employ monoclonal antibodies that are reactive to all isoforms. A preferred example of such an antibody is anti-human MEM-28, or Hl30. These antibodies may be brought into mixture and can then react with the cell surface membrane bound CD45 of white blood cells. Accordingly, CD45 detection with antibodies as mentioned, i.e. CD45 staining, may be used as counterstaining together with positive identification markers such as CD71 to exclude the white blood cell character of the cells of interest. Further information may be derived from suitable literature sources such as Schreier et al., 2017, Journal of translational medicine 15.1 (7), 6.

CD24 is a two-chain glycosylphosphatidylinositol (GPI)-anchored glycoprotein expressed at multiple stages of B-cell development, beginning with the bone marrow pro-B-cell compartment and continuing through mature, surface Ig positive B-cells. Plasma cell expression is typically very low or negative. CD24 is also expressed on the majority of B-lineage acute lympho-blastic leukemias, B-cell CCLs and B-cell non-Hodgkin’s lymphomas. CD24 may play a role in regulation of B-cell proliferation and maturation, and control of autoimmunity. Human CD24 has an expression pattern similar to homologous protein of a murine heat-stable antigen (HAS). HAS is a glycoprotein of a mouse, which is connected to cell membrane by fixed glycosylphos phatidyl-inositol (GPI) and composed of 31 amino acids in total. Among these amino acids, 16 amino acids are Ser, Thr and ASn residues that can be O-glycosylated and N-glycosylated (see Kay al., 1991, J Immunol, 147:1412-1416). The potential O-glycosylation sites are mainly located in the N-terminal and C-terminal region of CD24. CD24 is therefore expected to have a dumbbell like shape. Glycosylation of CD24 is assume to depend on cell types. The glycosylated CD24 has a wide range of molecular weight of 35 kDa-70 kDa (see Kristiansen et al., 2004, J Mol Histology 35:255-262). Unlike mouse HAS, human CD24 is typically not expressed in erythrocytes and thymocytes and only found in early stage B-lymphocytes (Kay et al., 1991, J Immunol, 147: 1412-1416). Human CD24 has been used as an early stage B-cell marker. CD24 can also be used as a marker for epidermal cells of the kidney and the brain under the developmental stage. A CD24 knock out mouse had no other functional defect but B-lymphocyte development (see Nielsen et al., 1997, Blood 89:1245-1258; Shirasawa et al., 1993, Dev Dyn 198: 1-13) indicating that CD24 is involved in the proliferation and maturation of pro B-lymphocytes. For the detection of CD24 any suitable antibody or other binding entity may be used. It is preferred to employ monoclonal antibodies. Examples of such antibodies include clone ML5 and M1/69.

CD133, also known as prominin-1, is a glycoprotein known to be expressed by immature hematopoietic stem cells but not mature blood cells. CD133 is, in particular, expressed in hematopoietic stem cells, endothelial progenitor cells and neural stem cells. CD133 was found to be present or enriched in cell populations found in several human solid tumors, such as colon carcinoma, melanoma and brain tumors (for example, glioblastoma). CD133 is a putative marker for cancer stem cells, i.e. cancer cells possessing stem-cell like characteristics such as an ability to differentiate in to multiple-cell types, and the ability to give rise to new tumors. CD133 protein was also found to be localized to membrane protrusions and to be often expressed on adult stem cells. One proposed function for CD133 is maintenance of “stemness” through suppression differentiation. CD133 absence and presence typically allows stratification between endothelial progenitor and mature endothelial cells. For the detection of CD133 any suitable antibody or other binding entity may be used. It is preferred to employ monoclonal antibodies. Examples of such antibodies include 13A4 and ab19898.

CD31 or PECAM-1 (Platelet endothelial cell adhesion molecule-1) is an inhibitory co-receptor involved in regulation of T cell and B cell signaling by a dual immunoreceptor tyrosine-based inhibitory motif (ITIM) that upon associated kinases-mediated phosphorylation provide docking sites for protein-tyrosine phosphatases. CD31 is expressed ubiquitously within the vascular compartment and is located mainly at junctions between adjacent cells. CD31 is a multifunctional molecule with diverse roles in modulation of integrin-mediated cell adhesion, transen-dothelial migration, angiogenesis, apoptosis, negative regulation of immunoreceptor signaling, autoimmunity, macrophage phagocytosis, IgE-mediated anaphylaxis and thrombosis. It is one of key regulatory molecules in vascular system. CD31 is further found on endothelial cells and neu-trophils and has been shown to be involved in the migration of leukocytes across the endothelium. CD31 consists of a single chain molecule comprising 6 Ig-like extracellular domains, a short transmembrane segments and a cytoplasmic tail containing two ITIMs. The structure of CD31 is expressed exclusively and constitutively on cells at the blood vessel interface. CD31 has also been implicated in the inflammatory process and an anti- CD31 monoclonal antibody has been reported to block in vivo neutrophil recruitment (see Nakada et al., 2000, .J. Immunol., 164: 452-462). It is, in certain embodiments, envisaged to determine, in order to detect, diagnose, monitor or prognosticate a pathologic status in a subject, the presence of the marker CD31 on a cell. In specific embodiment, the presence is detected, diagnosed, monitored or prognosticated on an endothelial cells, providing evidence of vascular damage. Furthermore, polyploidy and nuclear heterogeneity of such cells is associated with tumor growth (further information may be derived, for example, from Hida et al., 2004, Cancer research, 64,22: 8249-8255). For the detection of CD31 any suitable antibody or other binding entity may be used. It is preferred to employ monoclonal antibodies. Examples of such antibodies include WM59 and HEC7.

VAV1 is a protooncogene for the Dbl family of guanine nucleotide exchange factors for the Rho family of GTP binding proteins. VAV1 is assumed to be functional in hematopoiesis and possibly plays a role in B- and T-cell development and activation. Further details may be derived from suitable literature sources such as Bustelo et al., 1993, Cell growth & differentiation, 4(4), 297-308 or Fray et al., 2020, Journal of Cell Science. For the detection of VAV1 any suitable antibody or other binding entity may be used. It is preferred to employ monoclonal antibodies. Examples of such antibodies include 2E11 and ZV003.

The Kell blood group protein, also known as CD238, is a 93 kDa transmembrane zinc-dependent endopeptidase, which is assumed to be responsible for cleaving enthelin-3. This protein is a type II transmembrane glycoprotein. The encoding gene comprises several alleles, which leads to the presence of a highly polymorphic group of Kell blood group antigens (the antigens are defined as peptides comprised in the Kell blood group protein). The detection of the Kell blood group protein may be performed with an antibody, preferably a conjugated antibody such as A-10, which may be conjugated with phycoerythring or FITC, AlexaFluor 680 or 790. Further details may be derived from suitable literature sources such as Chen et al., 2009, Proceedings of the National Academy of Sciences, 106.41 (9): 17413-17418. For the detection of CD238 any suitable antibody or other binding entity may be used. It is preferred to employ monoclonal antibodies. Examples of such antibodies include OTI8E1, ETI5E6 and BRIC203.

In further embodiments, the presence of nucleic acids in cells of the subject’s sample is determined, preferably by methods as defined above, e.g. by staining the cells with suitable DNA dyes as mentioned above. In specific embodiments, the presence of nucleic acids in cells of the subject’s sample is determined in addition to CD71 and/or GPA, or optionally in addition to one or more of CD44, CD45, CD24, CD133, CD31, VAV1 and Kell blood group.

The biochemical markers may, in specific embodiments, be found on cells, e.g. erythroblasts or circulating tumor cells (CTCs), in combinations and/or amounts as derivable from the following Table 1. Accordingly, in particularly preferred embodiments the detection of the markers and marker combinations in association with the indicated amounts allows a clear detection of the cell types mentioned in the left hand column. Additional details may further be derived from the Examples, which describe tests and experiments envisaged by the present invention as general approaches.

TABLE 1

Presence of markers on specific cells according to the invention

| | | |
|---|---|---|
| Cell Type | Marker combination | Description |
| Normoblast | CD71 high/GPA high***/CD45 neg/CD44 neg or CD44 dim*/ DAPI high | Definitive erythroid phenotype |
| Immature erythroblast | CD71- or CD71dim/GPA high/CD45neg or CD45dim/CD44 high/DAPI high/ | Early erythroid phenotype |
| epithelial CTC | EpCam dim or EpCam high/CK neg/CD45neg/ DAPI dim to high | CTC with acquired epithelial character? Very rare group ? |
| epithelial CTC | EpCam neg/CK high/CD45-/ DAPI low** to high | Most common type reported/Group 1 or 4 |
| epithelial CTC | EpCam high/CK high/CD45neg/ DAPI low to high | Lower frequent Group 1 or 4 |
| EMT**** CTC | EpCamneg or Epcam dim/Ckneg/ Vimentin high/CD45 neg/ DAPI low or high | CTCs in Epithelial to mesenchymal transitions/Group 2 |
| Dead Epithelial CTC | EpCam neg to high/Ck high/CD45neg/DAPI dim | Group 3 or 4, final stage apoptosis |

* dim: slightly above background signal
** low: clearly detectable yet low
*** high: significantly above average
**** EMT: epithelial to mesenchymal transition

In a preferred embodiment the determination of the presence of one or more phenotypical markers as defined above in combination with one or more biochemical markers as defined above, e.g. in Table 1, may advantageously be used for the differential identification of cells in a subject’s sample and thus of a pathologic status in a subject’s bone marrow leading a specific bone marrow associated disorder. For example, the identification of aberrant erythroblasts showing an increased intranuclear and intracellular ploidy and simultaneously the identification of a CD71⁺ (positive) and/or GPA⁺ (positive); and CD45⁻ (negative) biochemical status in said erythroblasts in the sample may be seen as indicative for a bone marrow disorder such as myelodysplastic syndrome/acute myeloid leukemia, lymphomas, essential thrombocythemia or diabetes mellitus. In further preferred embodiments, the aberrant erythroblasts showing an increased intranuclear and intracellular ploidy may additionally show a nuclear budding and/or a lobulation phenotype and/or an internuclear bridge and/or two or more nuclei in a single cell and/or either (i) a megaloblast appearance or (ii) a macronormoblast appearance. In further specific embodiments, an aberrant erythroblast may show, in addition to a CD71⁺ (positive) and/or GPA⁺ (positive); and CD45⁻ (negative) biochemical status a CD44⁺ (positive), VAV1⁺ (positive), Kell blood group protein⁺ (positive) biochemical status. Some biochemical markers are redundant with respect to their diagnostic relevance on certain cell types, or may have overlapping diagnostic relevance. In such situations, which are derivable from the general description herein, they may be employed for detection of erythroblasts or CTCs or may not. For example, the use of biomarkers Kell blood group protein or VAV1 is considered optional and may be used as overlapping diagnostic tool. The decision whether such markers are used may be made dependent on the diagnostic setup, the availability of detecting agents, the specific apparatus used, the choice of staining methods etc. Corresponding parameters and cut-offs would be known to the skilled person.

In further embodiments, the presence of a further group of additional biochemical or molecular markers is envisaged. These additional markers comprise EpCam and cytokeratin. These markers may be determined individually together with CD71 and/or GPA or any one the group of CD44, CD45, CD24, CD133, CD31, VAV1, and Kell blood group protein as mentioned above, or they may be determined as differential group in combination with CD71 and/or GPA, CD44, CD45, CD24, CD133, CD31, VAV1, and Kell blood group protein e.g. EpCam plus CD71 and/or GPA, or cytokeratin plus CD71 and/or GPA; or EpCam plus CD71 and/or GPA plus any 1, 2, 3, or 4 items of the marker group CD44, CD45, VAV1, Kell blood group protein; or cytokeratin plus CD71 and/or GPA plus any 1, 2, 3, or 4 items of the marker group CD44, CD45, CD24, CD133, CD31, VAV1, Kell blood group protein; or EpCam plus cytokeratin plus CD71 and/or GPA; or EpCam plus cytokeratin plus CD71 and/or GPA plus any 1, 2, 3, or 4 items of the marker group CD44, CD45, CD24, CD133, CD31, VAV1, Kell blood group protein. Particularly preferred is the combination of CD71 and/or GPA plus EpCam plus CD45.

In certain embodiments it is additionally envisaged to use agents which are capable of detecting said marker(s), e.g. specific antibodies, as cocktails or mixtures of agents for all cells in a sample. Accordingly, a differential detection of erythroblasts and/or CTCs may become possible through such a multiplex approach. To increase specificity it is preferred to use diagnostically redundant or overlapping markers, i.e. their detection, in these multiplex approaches. This may, in certain embodiments, be implemented via the employment of different stains or dyes, which can be optically distinguished. It is further envisaged to modify said cocktails according to necessity and the cells which should be detected, e.g. in accordance with the information provided in Table 1.

EpCam, which is also known as epithelial cell adhesion molecule or CD326, is transmembrane glycoprotein mediating Ca²⁺-independent homotypic cell-cell adhesion in epithelia. It is also involved in cell signaling, migration, proliferation, and differentiation. EpCam expression is described for cancer cells, in particular colon carcinomas. Epcam cell surface antigen may be detected by conjugated antibody binding, preferably fluorescence dye labelled antibodies. The detection of EpCam expression of circulating tumor cells is considered to indicate the nature of cells to be epithelial progenitor-like cells or the EpCam expression was acquired during circulation or distant homing events. The detection of EpCam positive blastoid cells in healthy donors alludes to circulating epithelial progenitor cells and in case of mature cells alludes to circulating epithelial cells, such as squamous type or columnar type cells. Further information on the detection of EpCam may be derived from suitable literature sources such as Allard et al., 2004, Clinical cancer research, 10.20, 6897-6904.

Cytokeratin, which is also known as CK, is a keratin protein typically found in the intracytoplasmic cytoskeleton of epithelial tissue. Cytokeratins have been described as squamous keratins, belonging to the HMWCK family, or simple keratins, belonging to the LMWCK family. They are further subdivided into basic CKs and acidic CKs. There are up to at least 20 CK-types. In preferred embodiment, the detection of cytokeratin is performed with an antibody, e.g. a monoclonal IgG or any other suitable antibody form. It is further preferred that the antibody detects more than one family member of the family of cytokeratins. Accordingly, a pan-cytokeratin antibody, e.g. monoclonal antibody C-11, may be used. In alternative embodiments a mixture or combination of antibodies binding to different cytokeratin types may be employed. For example, the combination may comprise an antibody binding to Cytokeratin 1, e.g. LHK1, and/or Cytokeratin 3, e.g. AES, and/or Cytokeratin 4, e.g. SN74-03, and/or Cytokeratin 5, e.g. EP1601Y, and/or Cytokeratin 6, e.g. SN71-07, and/or Cytokeratin 7, e.g. RCK105 or OV-TL12/30, and/or Cytokeratin 8, e.g. M20, and/or Cytokeratin 10, e.g. DE-K10, and/or Cytokeratin 15, e.g. LHK15, and/or Cytokeratin 16, e.g. LL025, and/or Cytokeratin 17, e.g. E3, and/or Cytokeratin 18, e.g. DC10, and/or Cytokeratin 19, e.g. 4E8, and/or Cytokeratin 20,e.g. Ks20.8 and/or an antibody, e.g. monoclonal or polyclonal, binding to any other Cytokeratin.

In specific embodiments the antibody is conjugated to suitable labels such as HRP, FITC, Alexa Fluor, pycroerythrin etc. Cytokeratin detection typically requires fixation and permeabilization of the cells due to the intracellular expression of the filament. Depending on the location or cancer type, different cytokeratin types may be used. In further embodiments, in particular if the cancer type is not known, a pan-cytokeratin antibody cocktail may be employed. Cytokeratin is often used as single epithelial marker in circulating CD45⁻ (negative) cells in the presence of a nucleus. However, lowered specificity when compared to EpCam⁺/CD45⁻ phenotypes suggests expression of cytokeratins by other rare cells. A usually employed standardized test using the cytokeratin/CD45/DAPI phenotype is based on the CellSearch system as disclosed in Allard et al., 2004, Clinical cancer research, 10.20, 6897-6904.

In further embodiments, the presence of an additional group of biochemical or molecular markers is envisaged. Its use is optional. In certain embodiments, it is advantageous to also test these markers. This additional group of markers comprises vimentin. This group of markers may be determined individually together with CD71 and/or GPA or any one of the group of CD44, CD45, CD24, CD133, CD31, VAV1, Kell blood group protein, EpCam, and cytokeratin as mentioned above, or it may be determined as differential group in combination with CD71 and/or GPA, CD44, CD45, CD24, CD133, CD31, VAV1, Kell blood group protein EpCam, and cytokeratin, e.g. vimentin plus CD71 and/or GPA; or vimentin plus CD71 and/or GPA plus any 1, 2, 3, 4, 5, or 6 items of the marker group CD44, CD45, CD24, CD133, CD31, VAV1, Kell blood group protein, EpCam, cytokeratin.

Vimentin which is a type III intermediate filament (IF) protein, which is typically expressed in mesenchymal cells. Vimentin is thus an intracellular filament similar to cytokeratin. It comprises a central alpha-helix, capped on each end by non-helical amino and carboxy domains. Vimentin plays a significant role in supporting and anchoring the position of the organelles in the cytosol. It is, in particular, responsible for maintaining cell shape, integrity of the cytoplasm, and stabilizing cytoskeletal interactions. Vimentin is typically used as sarcoma tumor marker. Vimentin is preferably detected by fluorescence dye conjugated antibodies. The presence of vimentin⁺/CD45⁻ phenotypes typically suggests tumor cells in transition from epithelial to mesenchymal cell type thus being termed epithelial to mesenchymal transition or EMT-CTC. This find would allow for a a certain prognosis, which is assumed to be negative. Vimentin is assumed to be expressed in many normal non-hematpoietic cell types, making a clear distinction as tumor cell challenging. Further details may be derived from suitable literature sources such as Poliou-daki et al., 2015, BMC cancer, 15.1, 399.

In further embodiments, the determination of the presence of aberrant erythroblasts in a subject’s bodily fluid sample additionally comprises ascertaining whether cells the subject’s sample are present in the form of cell clusters. The term “cell cluster” as used herein relates to groups of two or more erythroblasts in the fluid sample analysed. These groups of cells originate from cell division and thus constitute even-numbered groups of cells. The number of clustered cells may be 2, 4, 6, 8, 10, 12, 14, 16, 18 to 20, preferably 2, 4, 6, 8, 10 cells. In additional embodiments, the determination of the presence of aberrant erythroblasts in a subject’s bodily fluid sample additionally comprises ascertaining whether cells the subject’s sample are present in the form of cell aggregates. The term “cell aggregate” as used herein relates to groups of three or more erythroblasts in the fluid sample analysed. These groups of cells originate from cell clumping events and thus constitute even-numbered or odd-numbers groups of cells. The number of aggregated cells may be 3, 4, 5, 6, 7, 8, 9 cells etc., preferably 3 cells.

In further preferred embodiments, the determination the presence of aberrant erythroblasts in a subject’s bodily fluid sample comprises a morphological analysis of cells in said sample. The term “morphological analysis” as used herein means that cells are observed with suitable optical or electronic means so that their form, e.g. geometric form, geometric irregularity etc.; shape; diameter; size, e.g. cellular area; form and size and position of subcellular entities, e.g. the nucleus such as nucleus area, perimeter or form; ratio of cellular or subcellular entities, e.g. nucleus/cytoplasm ratio, behaviour with respect to certain signals or environmental conditions; adherence behaviour with respect to neighbouring cells and other microbiological or histological parameters known to the skilled person can be determined. The term geometric form includes, for example, additional geometric description parameters such as perimeter, circularity, eccentricity, elongation, roundness, convexity, or nuclear eccentricity. Accordingly, the term “morphological analysis” may, in certain embodiments, also include the nuclear morphology of cells. Typically, a morphological analysis is performed with the help of a microscope or microscope system, preferably digital microscopy systems. Alternatively, the morphological analysis may be performed with automatic analyzers. Envisaged analysing systems include Cellavision DM96, Diffmaster Octavia, Cobas M511 analyzer. Further details are know to the skilled person or can be derived from suitable literature sources such as Merino et al., 2018, Intl. Journal of Laboratory Hematology, 40 (1), 54-61. In preferred embodiments, the cell analysis is conducted with automated microscopy systems such as the Perkin Elmer Operetta High content screening system, or the Merck Amnis image stream technology.

Further envisaged is the use of suitable software programs, which assist the morphological analysis, in particular image analysis programs. Such programs may further be combined with artificial intelligence systems or neural networks, e.g. DCNNs etc. Suitable examples of such programs include Imaris, CellProfiler, Kaleido, Columbus, image analysis software packages from Zeiss, e.g. ZEN, or Olympus, which are typically associated to corresponding microscope systems. Particularly preferred are individual software solutions based on common open source elements

Morphological analyses of cells according to the present invention are, in certain embodiments, also accompanied by cell staining procedures. Such cell staining may lead to the staining of one or more cellular compartments or elements. For example, the staining may be a nucleic acid staining as described herein, e.g. based on the use of DAPI etc., which allows to detect cellular regions with high nucleic acid concentration. An exemplary method is the Giemsa staining, which makes use of a mixture of methylene blue, eosin, and Azure B.

A preferred staining method is the May-Grünwald-Giemsa (MGG) staining which is typically used to stain cell nuclei. The method makes use of the dyes methylene blue and eosin Y or eosin B. The eosin dye typically stains basic areas within the cells such as e.g. DNA binding proteins. Further envisaged is a combination of May-Grünwald staining with Giemsa staining, which is called Pappenheim staining.

Further staining approaches may be used to detect other cellular compartments such as membranes. Examples of suitable stains are cell brite membrane stains, concavalin A, wheat germ agglutinin (WGA) conjugates, 1,6-Diphenyl-1,3,5-hexatriene, Biotin-DHPE, Biotin-X-DHPE, DiA 4-(4-Dihexadecylaminostyryl)-N-methylpyridinium iodide), DiB, DiD, or 1,1′-dioctadecyl-3,3,3′,3′-tetramethylindocarbocyanine.

In a central embodiment of the present invention, the morphological analysis of cells comprises or allows for classification of cells, in particular erythroblasts, in the sample into different classes. The corresponding parameter definition may accordingly be used in automatic systems or software programs to distinguish between the below defined cell classes, or may be provided to an expert to perform the classification according to the definition. Also envisaged is the use of corresponding training data for a machine learning or AI approach, wherein neural networks are trained in order to detect and classify cells in accordance with provided definitions. Corresponding cell images may accordingly be obtained and classified to be used as training data for an AI-approach, which is also envisaged as part of the present invention; or they may be stored in databases, e.g. on cloud-based servers, for future or parallel use.

The classification comprises the following distinction of erythroblasts derived from a bodily fluid sample, e.g. blood sample:
class 1: in this class the bodily fluid sample comprises cells which are round or oval and which comprise one nucleus;
class 2: in this class the bodily fluid sample comprises cells which are round or oval and which comprise at least two nuclei;
class 3: in this class the bodily fluid sample comprises pairs of cells comprising a constriction; and
class 4: in this class the bodily fluid sample comprises aggregations of at least three round or oval cells with one or more nucleus or nuclei.

The above mentioned geometric parameters may be suitable encoded in autoed system. They can be defined, for example, in accordance with exemplary images and a definition of divergence or deviation from said exemplary form, e.g. by 10%, 20%, 30%, 35%.

In a further preferred embodiment, the above mentioned cell classes may further be divided into one or more subclasses according to the presence of certain cell types, nucleus diameter values, or cell diameters. The term “diameter” as used herein refers to the distance between the two most distant points on the perimeter of a cell, in particular of a cell image as obtained by microcopy or other cell imaging methods as described herein.

For class 1, the following sub-classification may be used:
class 1a, wherein the sample comprises normal erythroblasts with a diameter of about 6.5 µm to 12.4 µm with dense nucleus and high nucleus to cytoplasm ratio;
class 1b, wherein the sample comprises giant circulating erythroblasts of a diameter of >12.5 µm, of at least one low density nucleus in diameter of 6 to 10 µm and low nucleus to cytoplasm ratio;
class 1c, wherein the sample comprises megaloblasts with nucleocytoplasmic asynchrony and moderate to high density chromatin and high nucleus to cytoplasm ratio; and
class 1d, wherein the sample comprises macronormoblasts with no nucleocytoplasmic asynchrony, total condensation nuclei in a diameter of 4 µm to 6 µm and a low nucleus to cytoplasm ratio.

In addition, the ascertaining whether cells the subject’s sample are present in the form of cell clusters can be accompanied, preferably in an optional way, by determining the presence of vimentin or EpCam as described above.

Class 1a cells are morphologically or phenotypically normal erythroblasts. They typically have a diameter of about 6.5 µm to 12.4 µm. The cells show a dense nucleus and high nucleus to cytoplasm ratio. An aberrant behaviour of class 1a cells may be attributable to their quantity, in particular their quantity in a defined volume of a bodily fluid sample, e.g. blood sample. In certain embodiments, the number of class 1a cells may be at 1 x 10⁴ cells per ml of sample, e.g. a whole blood sample. According to preferred embodiments of the invention as defined herein, class 1a cells, when used as biomarker for bone marrow damage, have a clinical interpretation power by allowing a grading bone marrow damage into mild, moderate and severe damage, as explained herein.

Class 1b cells comprise giant circulating erythroblasts. These giant erythroblasts have a diameter of >12.5 µm. They typically have one low density nucleus in diameter of 6 to 10 µm and a low nucleus to cytoplasm ratio. These giant erythroblasts are considered as normal early erythroblasts, and therefore have a larger size in the range of about 12.5 to 25 µm and contain a nucleus with less dense chromatin when compared to class 1a cells, a relative low nuclear-cytoplasmic (N/C) ratio when compared to class 1 a cells and megaloblasts, yet a higher N/C ratio when compared to macronormoblasts.

Class 1c cells comprise megaloblastoid or megaloblast-like cells, i.e. cells which morphologically resemble megloblasts. The presence of these cells in a sample, e.g. blood sample, is typically considered as one indicative factor or as a cellular hallmark feature of myelodysplastic leukemia. According to the present invention, a cell of class 1c morphologically resembles a megaloblast, but is an aberrant erythroblast. A cell of class 1c typically comprises a large nucleus with heterogeneous chromatin density, preferably with a diameter exceeding 7.5 µm. Furthermore, the cells have a cellular appearance with high N/C ratios. This property allows to distinguish the class 1c cells from other giant erythroblasts, which typically exhibit a lower N/C ratio with less chromatin densification. According to the present invention the presence of class 1c cells in a bodily fluid sample, e.g. blood, is considered to be indicative for bone marrow neoplasms. They can, in certain embodiments, also be detected in samples of carcinoma patients.

Class 1d cells according to the present invention include large erythroblasts with a diameter of about 4 µm to 6 µm, which have the morphological aspect of a macro-normoblast. The class 1d cell typically shows a very small highly dense nucleus similar to the most matured stage, however with a large cytoplasm. The class 1d cells is considered aberrant in terms of erythropoiesis, in particular including certain impairments along the erythroblast maturation procedure. According to the present invention the presence of class 1d cells in a bodily fluid sample, e.g. blood, is considered to be indicative for bone marrow neoplasms. They can, in certain embodiments, also be detected in samples of carcinoma patients.

For class 2, the following sub-classification may be used:
class 2a, wherein the cells are bi-nucleated;
class 2b, wherein the cells contain at least 3 nuclei;
The nuclei of cells, which have 2 or 3 nuclei as in class 2, are either clearly distinguished from each other, e.g. present at different positions within the cell and/or not linked by any association structure or what seems to be ongoing nuclear division. The density of the nuclei or chromatin density is preferably identical or similar. The severity in aberration may be indicated by inequality of nuclei per cell with respect to size and chromatin density. The cells in this class may have the cellular morphology of any of the cells described in class 1.

Class 2 cells can generally be divided into bi- (class 2a) and multinucleated (class 2c) cells due to presumed differences in pathology. Binucleated cells (class 2a) represent sufficient morphological character as such and are generated by virtue of impairments in the cytokinesis process that may include a lack of cell ingression or a lack in cell stabilization. However, these cells may be diverse with respect to cell size and shape, the nucleus size distribution, distances between the two nuclei and nucleus density. The nucleic appearance in the class 2 cells is independent of the mitosis situation, i.e. the cells may show this phenotype in division or in completely separated cells. Class 2b cells typically share the same morphological description as class 2a cells apart from containing at least three nuclei. In class 2b cells, unintentional endomitosis is assumed to occur, thus constituting a pathological status causing the production of multiple chromosome sets in a single cell.

For class 3, the following sub-classification may be used:
class 3a, wherein the cell shows no nuclear bridge; and
class 3b, wherein the cell shows a nuclear bridge and wherein the nuclei show an inequality in size, shape and/or chromatin density.

The cells in this class may have the morphological peculiarity of a constriction. The term “constriction” as used herein refers to morphology which causes the cell to take on an asymmetric shape with a contraction at a certain point. The constriction phenotype is assumed to be connected to mitosis events in the cell.

The term “nuclear bridge” as used in the context of class 3 cells relates to a nuclear morphology, wherein the nucleus is enlarged in size and partially divided into two portions, which are still connected. The connection between these two portions has the form of a bridge or arch. The nuclear bridge may, in certain embodiments, be found at the zone of constriction between within the cell, i.e. at the zone where nuclear separation during mitosis takes place.

Accordingly, class 3 cells are, in comparison to the other classes of cells defined herein, morphologically distinguishable since they comprise a specific cell population undergoing mitosis, i.e. they represent a morphology of pairs of cells in cytokinesis. This interpretation is derived from the observation of paired cells showing cell membrane constriction, which excludes the possibility of two attached cells being aggregates. Class 3a cells typically present a rather normal morphology, which is similar to class 1a cells, yet being in division. In contrast to class 1a cells, class 3a cell are present in a bodily fluid sample, e.g. blood.

In contrast to class 3a cells, the class 3b cells typically represent extensive aberration indicating more dramatic damage as caused by cancers. This cell subclass shows paired cells seemingly in division with two unequal nuclei in size. The cells further show a nuclear bridging and changed chromatin density linked to the morphological feature of impaired division.

Class 4 cells occur in groups of at least three round or oval cells with one or more nucleus or nuclei. Class 4 cell thus have the morphological aspect of clusters of cells as defined herein. These cells are seemingly in division or constitute a breakaway of aggregated erythroblasts from an erythroid island within the bone marrow. The appearance of such cell types typically reflects physical damage of the bone marrow. Metastatic growth within the bone marrow is likely in case of a class 4 cells. In certain embodiments, calls 4 cells may further comprise aberrations. There is no difference between class 4 and 3b cells apart from the clustering or aggregations.

In particularly preferred embodiments the determination of the presence of aberrant erythroblasts in a subject’s bodily fluid sample additionally comprises the determination of the ratio of at least one of: (i) class 1a cells vs. class 1c/1d cells; (ii) class 1a cells vs. class 2 cells; and (iii) class 1a cells vs. class 3 cells, and of clusters of circulating tumor cells (CTCs). The ratio of cells according to certain classes as defined herein is determined by counting the cells within a specific volume of the bodily fluid sample mentioned herein, preferably within a specific volume of blood. The volume itself can suitably be chose and may depend on the sampling or sample preparation procedure. The present invention is based on the determination of ratio, thus envisaging the use of any suitable sample volume or size. It is preferred, for statistical reasons, to use larger sample volumes. It is further envisaged to perform the determination of the ratio with more than one sample of a patient. The cell counting may be performed with simple counting chambers or with the assistance of cell imaging systems or programs, e.g. as mentioned herein.

The term circulating tumor cell (CTC) as used herein relates to a cell that has shed into the vasculature or lymphatics from a primary tumor and is carried around the body in the blood circulation CTCs can become the seeds for subsequent growth of additional tumors, i.e. metastases, in distant organs. CTCs which are derived from tumors that include carcinomas, sarcomas, and melanomas, may be classified according to the expression of epithelial markers, or their size and apoptosis status. One class (group 1) of CTCs shows an intact, viable nucleus; the expression of EpCam and cytokeratins, which demonstrate epithelial origin; the absence of CD45, indicating the cell is not of hematopoietic origin; and their larger size, irregular shape or subcellular morphology. A further class (group 2) of CTCs is cytokeratin-negative, i.e. characterized by the lack of EpCam or cytokeratins, which may indicate an undifferentiated phenotype (circulating cancer stem cells) or the acquisition of a mesenchymal phenotype (EMT). A further group of CTCs (group 3) are apoptotic CTCs, which otherwise belong to group 1, i.e. are traditional CTCs that are undergoing apoptosis. A fourth group of cells (group 4) are small CTCs, which are cytokeratin-positive and CD45-negative, but with sizes and shapes similar to white blood cells. A fifth group of cells (group 5) are small or large cells, which are EpCam⁺ (positive) and negative for CK and CD45.

A circulating tumor cell cluster (CTC cluster) comprises two or more individual CTCs bound together. The CTC cluster may contain cells of any one of groups 1 to 5 of CTSs as defined herein. These clusters typically have cancer-specific biomarkers that identify them as CTCs. The identification of CTC clusters is considered indicative for late stage tumor evolution.

In a preferred embodiment the identification of at least one of (i) to (iv) in a subject’s sample: (i) an EpCam⁺ (positive) and CD45⁻ (negative) circulating tumor cell (CTC) in the sample (marker 1); (ii) a CTC cell cluster with a single cell diameter of about 6 to 20 µm of cells showing marker 1 in the sample (marker 2); (iii) EpCam⁻ (negative) and vimentin⁺ (positive) CTCs in the sample (marker 3), e.g. CTCs positive for the expression of vimentin and nucleus staining and negative for the expression of CD45. Such CTCs may be positive for EpCam expression. CTCs with said phenotypes are referred to as CTCs in epithelial to mesenchymal transition and abbreviated as EMT CTCs; and additionally of (iv) the presence of a ratio of at least one of (i) class 1a cells vs. class 1c/1d cells; (ii) class 1a cells vs. class 2 cells; and (iii) class 1a cells vs. class 3 cells of 50 or less to 0 is indicative for the malignancy of a solid tissue cancer.

A ratio of 50 or less to 0 as mentioned above may, in certain embodiments, include a ratio of about 50 to 0, about 45 to 0, about 40 to 0, about 35 to 0, about 30 to 0, about 25 to 0, about 20 to 0, about 15 to 0, about 10 to 0, about 5 to 0, about 3 to 0, about 2 to 0, or any other value (integer) between or below the mentioned values. The ratio, in particular, applies only in the presence of erythroid abnormality and indicates presence of the above and below mentioned diseases or disorders, in particular invasive cancer.

The term “solid tissue cancer” as used herein relates to localized solid tumors of different types including sarcomas, melanomas and carcinomas. Envisaged examples are colon carcinoma, prostate cancer, breast cancer, lung cancer, skin cancer, liver cancer, bone cancer, ovary cancer, pancreas cancer, brain cancer, head cancer, neck cancer, neuroblastoma, Wilm’s tumor, or retinoblastoma.

The term “malignancy” as used herein relates to malignancy in the context of tumors. This is typically characterized by anaplasia, invasiveness, and metastasis. Malignant tumors are also typically characterized by genome instability, so that cancers, as assessed by whole genome sequencing, frequently have between 10,000 and 100,000 mutations in their entire genomes. Malignant tumors usually show tumour heterogeneity, containing multiple sub-clones. They also frequently have reduced expression of DNA repair enzymes due to epigenetic methylation of DNA repair genes or altered microRNAs that control DNA repair gene expression.

In a further preferred embodiment the identification of class 1a cells present in an amount of about 10 to 500 cells per ml of the sample and of class 1b/1c/1d cells present in an amount of about 1 to 10 cells per ml in the sample is indicative for a mild bone marrow damage as defined herein above.

The term “1b/1c/1d” or any other combination with “/” between certain cell classes as used herein relates, when a value or number is involved, to a combination or sum of the cells of classes e.g. 1b, 1c and 1d in the sample, or of cell classes mentioned before and after the “/” sign.

In a further embodiment, the identification of class 1a cells present in an amount about 1000 to 5000 cells per ml of the sample and of class 1b/1c/1d cells present in an amount of about 10 to 50 cells per ml of the sample is indicative for a moderate bone marrow damage.

In a further embodiment, the identification of class 1a cells present in an amount greater than about 10 000 cells per ml of the sample and of class 1b/1c/1d cells present in an amount greater than about 10 cells per ml of the sample is indicative for a severe bone marrow damage.

The term “an amount of 1 to 10 cells per ml” as used herein means that about 1, 2, 3, 4, 5, 6, 7, 8, 9 or 10 cells are found in a ml of bodily fluid sample, e.g. a blood sample as defined herein. The term “an amount of 10 to 50 cells per ml” as used herein means that about 10, 20, 30, 40, or 50 cells or any value in between the mentioned values is found in a ml of bodily fluid sample, e.g. a blood sample as defined herein. The term “an amount of 10 to 500 cells per ml” as used herein means that about 10, 20, 30, 40, 50, 100, 150, 200, 250, 300, 350, 400, 450 or 500 cells or any value in between the mentioned values is found in a ml of bodily fluid sample, e.g. a blood sample as defined herein. The term “an amount of 1000 to 5000 cells per ml” as used herein means that about 1000, 1500, 2000, 2500, 3000, 3500, 4000, 4500 or 5000 cells or any value in between the mentioned values is found in a ml of bodily fluid sample, e.g. a blood sample as defined herein. The term “an amount of 0.5 to 10 cells per ml” as used herein means that about 0.5, 1, 2, 3, 4, 5, 6, 7, 8, 9 or 10 cells are found in a ml of bodily fluid sample, e.g. a blood sample as defined herein. Also, non-integers such as 1.5, 2.5 etc. are possible. The term “0.5 cells found in ml of bodily fluid sample” as used herein means that 1 cell may be found in 2 ml of bodily fluid, or 2 cells may be found in 4 ml of bodily fluid etc. Accordingly, “1.5 cells in a ml” means that 3 cells can be found in 2 ml etc.

In a particularly preferred embodiment, the method as described herein comprises the identification of a mild or moderate bone marrow damage as described above and the identification of marker 1, 2 and/or 3 as defined above and the identification of a ratio of 50 or less to 0 as defined above is indicative for a cancer treatment related bone marrow damage of non-invasive cancer or a dormant cancer disease, minimal residual cancer disease or micro-metastasis in the bone marrow related to invasive solid tissue cancer.

In a specific embodiment, the present invention hence envisages that the combination of features: a mild bone marrow damage is given due to the identification of class 1a cells present in an amount of about 10 to 500 cells per ml of the sample and of class 1b/1c/1d cells present in an amount of about 1 to 10 cells per ml of the sample and further either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for a dormant cancer disease, a minimal residual cancer disease or a micro-metastasis in the bone marrow related to invasive solid tissue cancer.

In a further specific embodiment, the present invention envisages that the combination of features: a moderate bone marrow damage is given due to the identification of class 1a cells present in an amount about 1000 to 5000 cells per ml of the sample and of class 1b/1c/1d cells present in an amount of about 10 to 50 cells per ml of the sample and further either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for a dormant cancer disease, a minimal residual or invasive minimal residual cancer disease or a micro-metastasis in the bone marrow related to progressive invasive solid tissue cancer.

In a further specific embodiment, the present invention envisages that the combination of features: a severe bone marrow damage is given due to the identification of class 1a cells present in an amount greater than about 10 000 cells per ml of the sample and of class 1b/1c/1d cells present in an amount greater than about 10 cells` per ml of the sample and further either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) andCD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for an actively developing micro-metastasis in the bone marrow related to an invasive solid tissue cancer.

According to the present invention class 1a quantification is linked to bone marrow damage. With respect to erythroblast aberration in the circulation, current methods are either not sufficiently sensitive or not equipped to discriminate finest morphological differences. According to the present invention, erythroblast concentration levels are associated with severe disease stage being in the range of 1 x 10⁴ to 2 x 10⁵ normoblasts per ml of bodily fluid sample, e.g. blood sample. In specific embodiments, the amount of class 1a cells may be linked to bone marrow damage according to the following Table 2.

TABLE 2

| | |
|---|---|
| Bone marrow damage | Class 1a frequency in cells per ml sample |
| Healthy | Approx.<17 per ml |
| Mild | Approx. 17 - 4999 |
| Moderate | Approx. 5x10³ - 9x10⁴ |
| Severe | Approx. >1x10⁵ |

At the same time number of class 1b/1c/1d cells may have an additional value for the determination of bone marrow damage. For example, in a mild bone marrow damage situation, the number of class 1b/1c/1d cells is rather low within a range of about 1 to 10 cells per ml of the sample.

Further, in a moderate bone marrow damage situation, the number of class 1b/1c/1d cells slightly elevated to about 10 to 50 cells per ml of the sample.

Further, in a severe bone marrow damage situation, the number of class 1b/1c/1d cells is similar to the number in the moderate bone marrow damage situation, namely greater than 10 cells per ml of the sample. However, the number may exceed also 50 cells per ml. Typically, the number will be in a range of about 10 to 50 cells, more preferably in range of about 10 to 20 cells.

For class 1c cells a cut-off of 2.1 cells per ml sample, e.g. blood, has been identified to indicate mild bone marrow damage according to the specifications and parameters of the detection system used, e.g. as exemplified and described in Example 1. It is envisaged that cut-offs as mentioned may change in accordance with systems and appartuses used, e.g. due to modified detection mechanisms, different resolutions etc. The skilled person would be able to adapt the currently mentioned cut-offs and values to further, e.g. future, systems by performing a comparison and/or calibration procedure. Moderate bone marrow damage in a subject, e.g. a cancer patients is, in certain embodiments, associated with a cell number in the range of about 10 to 50 cells per ml sample, e.g. blood. Severe bone marrow damage is, in specific embodiments, indicated by more than 20 cells per ml of sample, e.g. blood. A severe bone marrow damage is, in further embodiments, considered to be associated with the likelihood or presence of bone metastases.

In a further preferred embodiment, the identification of class 2a and class 2b cells in an amount of about 0.5 to 10 cells per ml of sample is indicative for a moderate bone marrow damage.

In yet another preferred embodiment, the identification of class 2a cells and class 2b cells in an amount of about 10 to 50 cells per ml of sample, is indicative for a severe bone marrow damage.

The class 2 cell type as defined above is considered specific towards severe disorders, such as leukemia, carcinomas, and anemia. Accordingly, the detection of one or more cells in the sample suggests bone marrow damage. In certain specific embodiments, a cut-off from about 0.3 to 0.6 cells per ml sample is considered indicative for bone marrow damage. This transforms into a minimal LOD of 0.5 to 1 cell per ml. Further details may be derived from Example 3, which also explains a possible calculation of the cut-off determination. The presence of an amount of class 2 cells above cut-off would indicate at least a moderate damage. A discrimination between multi (class 2b) and binucleation (class 2a) further, in specific embodiments, allows a discrimination between metastasis and leukemia or congenital dyserythropoietic anemia type II (CDAII). According to the present invention, the presence of only class 2a cells and CTCs above cut-off in both cases and consequently, in the absence of class 2b cells, indicates an externally inflicted bone marrow damage. The formation of both class 2a and 2b cells is subject to different pathologies. Without wishing to be bound by theory, multinucleation as represented by class 2b cells is assumed to be due to a cell intrinsic genetic factor due to a bone marrow-native pathology, as for example present in leukemia. Strict binucleation often relates to chemical perturbations of erythroid mitotic processes in particular erythroblast cytokinesis, for example induced externally by inflammatory tumor cell activity within the bone marrow. In contrast, in the presence of class 2a and class 2b cells, intrinsic bone marrow damage is considered to be given, which alludes to bone marrow disorders such as anemia and leukemias. In a further embodiment, the finding of class 2a cells and class 2b cells without CTCs rules out underlying bone metastasis. In further embodiments, the presence of class 2a cells and class 2b cells and the presence of EpCam+ cells refers to externally and internally derived bone marrow damage.

Accordingly, the identification of a moderate bone marrow damage as defined in the context of class 2 cells and in the absence of class 2b cells per ml of sample and the identification of marker 1, 2 and/or 3 as defined above and the identification of a ratio of 50 or less to 0 as defined above is indicative for the presence of a progressive micro-metastasis in the bone marrow related to an invasive solid tissue cancer. The term “progressive micro-metastasis” as used herein relates to a systemic spread from the bone marrow by means of a micro-metastasis that however may not develop into a bone metastasis.

In a further embodiment, the identification of a severe bone marrow damage defined in the context of class 2 cells in the absence of class 2b cells per ml of sample and the identification of marker 1, 2 and/or 3 as defined above and the identification of a ratio of 50 or less to 0 as defined above is indicative for a bone metastasis related to invasive solid tissue cancer or primary bone cancer.

In a specific embodiment, the present invention hence envisages that the combination of features: a moderate bone marrow damage is given due to the identification of class 2a and class 2b cells in an amount of about 0.5 to 10 cells per ml of the sample and further class 2b cells are absent per ml of sample and either EpCam⁺ (positive) and CD45- (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for the presence of an active micro-metastasis in the bone marrow related to an invasive solid tissue cancer. The term “active micro-metastasis” or “actively developing micro-metastasis” as used herein relates to a cancerous state, which is developing into a metastasis.

In a further specific embodiment, the present invention hence envisages that the combination of features: a severe bone marrow damage is given due to the identification of class 2a cells and class 2b cells in an in an amount of about 10 to 50 cells per ml of sample and further class 2b cells are absent per ml of sample and either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative indicative for a bone metastasis related to invasive solid tissue cancer or primary bone cancer.

In a further preferred embodiment, the identification of class 3a cells in an amount of about 1 to 10 cells per ml of sample is indicative for a moderate bone marrow damage.

In yet another preferred embodiment, the identification of class 3a cells in an amount of about 10 to 50 cells per ml of sample and/or class 3b cells in an amount of about 1 to 10 cells per ml of sample is indicative for a severe bone marrow damage.

Class 3a cells may, according to certain embodiments of the present invention, occur in individuals with bone marrow damage unrelated to leukemia or cancer at very low concentrations or amounts. Further details may be derived, for example, from Example 6. The presence of this cell class is typically considered as phenomenon of bone marrow damage of various etiologies.

In contrast to the class 3a cells, class 3b cells represent extensive genetic aberration indicating more dramatic damage as could be caused by cancers. Accordingly, class 3b cells indicate severe bone marrow damage, whereas class 3a cells rather indicate milder damage.

Accordingly, the identification of a moderate bone marrow damage as defined in the context of class 3 cells and in the absence of class 2b cells per ml of sample and the identification of marker 1, 2 and/or 3 as defined above and the identification of a ratio of 50 or less to 0 as defined above is indicative for the presence of a progressive micro-metastasis in the bone marrow related to an invasive solid tissue cancer.

In a further embodiment, the identification of a severe bone marrow damage defined in the context of class 3 cells in the absence of class 2b cells per ml of sample and the identification of marker 1, 2 and/or 3 as defined above and the identification of a ratio of 50 or less to 0 as defined above is indicative for a bone metastasis related to invasive solid tissue cancer or primary bone cancer.

In a specific embodiment, the present invention hence envisages that the combination of features: a moderate bone marrow damage is given due to the identification of class 3a cells in an amount of about 1 to 10 cells per ml of sample and further class 2b cells are absent per ml of sample and either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for the presence of a progressive micro-metastasis in the bone marrow related to an invasive solid tissue cancer.

In a further specific embodiment, the present invention hence envisages that the combination of features: a severe bone marrow damage is given due to the identification of class 3a cells in an amount of about 10 to 50 cells per ml of sample and/or class 3b cells in an amount of about 1 to 10 cells per ml of sample and further class 2b cells are absent per ml of sample and either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative indicative for a bone metastasis related to invasive solid tissue cancer or primary bone cancer.

In a further preferred embodiment, the identification of class 4 cells in an amount of a least one cell per ml of sample is indicative for a severe bone marrow damage.

The appearance of class 4 cells typically reflects severe damage of the bone marrow above cut-off. In further embodiments metastatic growth within the bone marrow can be diagnosed or detected in case of class 4 cells in the bodily fluid sample.

Accordingly, in a further embodiment, the identification of a severe bone marrow damage defined in the context of class 4 cells in the absence of class 2b cells per ml of sample and the identification of marker 1, 2 and/or 3 as defined above and the identification of a ratio of 50 or less to 0 as defined above is indicative for indicative for a bone metastasis related to invasive solid tissue cancer or primary bone cancer.

In a further specific embodiment, the present invention hence envisages that the combination of features: a severe bone marrow damage is given due to the identification of class 4 cells in an amount of a least one cell per ml of sample and further class 2b cells are absent per ml of sample and either EpCam⁺ (positive) and CD45⁻ (negative) circulating tumor cell (CTC) are found in the sample; and/or CTC clusters of a size of about 6 to 20 µm of cells which show the biochemical marker combination of EpCam⁺ (positive) and CD45⁻ (negative) are found in the sample; and/or EpCam⁻ (negative) and vimentin⁺ (positive) CTCs are found in the sample, and a ratio of at least one of (i) class 1a cells vs. class 1c/1d cells; (ii) class 1a cells vs. class 2 cells; and (iii) class 1a cells vs. class 3 cells of 50 or less to 0 is identified; is indicative for a bone metastasis related to invasive solid tissue cancer or primary bone cancer.

In a further aspect the present invention relates to the use of an aberrant erythroblasts as a marker for diagnosing, detecting, monitoring or prognosticating a pathologic status in a subject’s bone marrow. The aberrant erythroblast is an erythroblast as defined herein above. In preferred embodiments the aberrant erythroblast is a class 1c, 1d, 2a, 2b, 3a, 3b or 4 cell as defined herein. The pathologic status is typically connected to a disease or disorder as defined herein.

In a preferred embodiment, wherein said aberrant erythroblast are identified as mentioned above. In specific embodiments, erythroblasts are observed, counted per ml sample and their classification according to the herein disclosed classification scheme (class 1a to class 4) is performed. Upon such classification and counting the detection of certain numbers and ratios of erythroblasts may be used for the inference of the pathologic status in a subject’s bone marrow. For example, the observation and classification may yield the following numbers and scenarios:

Class 1a cells are present in an amount of about 10 to 500 cells per ml in the sample and class 1b/1c/1d cells are present in an amount of about 1 to 10 cells per ml in the sample. In this situation the detected aberrant erythroblasts may be used to infer the identification of a mild bone marrow damage.

Class 1a cells are present in an amount about 1000 to 5000 cells per ml in the sample and class 1b/1c/1d cells are present in an amount of about 10 to 50 cells per ml in the sample. In this situation the detected aberrant erythroblasts may be used to infer the identification of a moderate bone marrow damage.

Class 1a cells are present in an amount greater than about 10 000 cells per ml in the sample and class 1b/1c/1d cells are present in an amount greater than about 10 cells per ml in the sample. In this situation the detected aberrant erythroblasts may be used to infer the identification of a severe bone marrow damage.

Class 2a and class 2b cells are present in an amount of about 0.5 to 10 cells per ml of sample. In this situation the detected aberrant erythroblasts may be used to infer the identification of a moderate bone marrow damage.

Class 2a cells and class 2b cells are present in an amount of about 10 to 50 cells per ml of sample. In this situation the detected aberrant erythroblasts may be used to infer the identification of a severe bone marrow damage.

Class 3a cells are present in an amount of about 1 to 10 cells per ml of sample. In this situation the detected aberrant erythroblasts may be used to infer the identification a moderate bone marrow damage.

Class 3a cells are present in an amount of about 10 to 50 cells per ml of sample and/or class 3b cells are present in an amount of about 1 to 10 cells per ml of sample. In this situation the detected aberrant erythroblasts may be used to infer the identification a moderate bone marrow damage.

Class 4 cells are present in an amount of a least one cell per ml of sample. In this situation the detected aberrant erythroblasts may be used to infer the identification a severe bone marrow damage.

The detection of these scenarios may be either mutually exclusive or at least partially overlapping. Thus, according to specific embodiments of the present invention class 1a cells may be found in all situation, i.e. class 1a cells may be present in combination with class 1b, 1c, 1d, 2, 3 or 4. The presence of class 1a cells thus indicates healthy or diseased states, depending on the presence of other classes etc. as outlined herein. Class 1b cells may be found in combination with class 1a cells, or in combination with class 1a, class 2, class 3, or class 4 cells. In a situation where class 1a and class 1b cells are found without the other classes excluding quantitative abnormality, the subject is considered to be healthy. In contrast, if class 1b cells is found together with class 1a and class 2; or together with class 1 a and class 3 and class 4 cells, the subject is diseased. Further, class 1a and class 1b cells may be found together with class 2 cells, which also indicates a disease state. Further, class 1a and class 3 cells may be found, also indicating a disease state. Further class 4 cells may be found together with class 1a and class 2 and/or class 3 cells, indicating a disease state. Further, the presence of 1c cells and/or 1d cells and class 2 cells indicates a disease state. Further, class 3 and/or class 4 cells found together with class 1c and/or class 1d cells indicates a disease state.

In certain embodiments said aberrant erythroblasts are used in combination with additional markers as defined above. For example, the aberrant erythroblasts are used in combination with the marker circulating tumor cell (CTC). In preferred embodiments, the present invention envisages the identification of aberrant erythroblasts as defined above, as well as the identification of CTCs showing certain biochemical markers. For example, the CTCs may be EpCam⁺ (positive) and CD45- (negative). Alternatively, the CTCs may have CTC cell clusters of wherein the single cells have a diameter of about 6 to 20 µm and be EpCam⁺ (positive) and CD45⁻ (negative). In a further alternative, the CTCs may be EpCam⁻ (negative) and vimentin⁺ (positive) CTCs.

On the basis of the described uses it may be inferred that the pathologic status is malignant solid tissue cancer such as a non-invasive solid tissue cancer in the presence of mild or no bone marrow damage. Further details are provided herein above in the context of the methods of the present invention. These details are included as additional embodiments also for the use claims.

In a further aspect the present invention relates to a composition for diagnosing, detecting, monitoring or prognosticating a pathologic status in a subject’s bone marrow, comprising means for the determination of the presence of (i) CD71 and/or GPA, (ii) CD45, (iii) nucleic acids of rare circulating cells, and at least one of (iv) or (v): (iv) EpCam and (v) vimentin, in a subject’s sample. In further embodiments, the composition may also comprise means for the detection of the presence of one, two, three or all of CD44, CD24, CD133 and CD31.

The term “means for determination” as used herein relates to a means as defined herein above in the context of CD71, GPA, CD45, EpCam, Vimentin, CD44, CD24, CD133 and CD31 and nucleic acids, preferably an antibody binding to CD71, e.g. as defined herein above, and/or an antibody binding to GPA, e.g. as defined herein above, and/or an antibody binding to CD45, e.g. as defined herein above, and/or an antibody binding to EpCam, e.g. as defined herein above, and/or an antibody binding to Vimentin, e.g. as defined herein below, and/or an antibody binding to CD44, e.g. as defined herein above, and/or an antibody binding to CD24, e.g. as defined herein above, and/or an antibody binding to CD133 e.g. as defined herein above, and and/or an antibody binding to CD31, e.g. as defined herein above, as well as a dye which stains nucleic acids such as DAPI or Hoechst 33342 etc. Means for the detection of EpCam and vimentin have been defined herein above, preferably an antibody, which binds EpCam, and an antibody which binds vimentin. In a specific embodiment, the composition comprises means for the detection of EpCam which are anti-EpCam antibodies MH99 and/or VU-1D9. In further specific embodiments, the composition comprises means for the detection of Vimentin which are, for example, anti-Vimentin antibodies V9, J144, RV202, SP20 and/or VI-01.

The term “means for the determination nucleic acids of rare circulating cells” as used herein relates to a dye which stains the nucleic acid of a rare circulating cell. The term may further extend to means for obtaining said nucleic acids, e.g. means for enriching said cells or for specifically binding nucleic acids of these cells. Means for the determination nucleic acids of rare circulating cells may, in certain embodiments, further or alternatively include means to measure chromosomal abnormality, such as polyploidy and aneuploidy. Such means include components for FISH or the like, or components for LCFM such as Hoechst 33342 or the like. Corresponding information would be known to the skilled person or can be derived from suitable literature sources such as Godek, 2018, Methods Cell Biol.; 144: 15-32; and Gomes et al., 2018, Cell Div, 13:6. The composition of the present invention is preferably a diagnostic composition. It may be formulated in accordance with routine procedures known to the skilled person. For example, diagnostic compositions may comprise sterile isotonic aqueous buffer. Where necessary, the composition may also include a solubilizing agent.

The composition may further comprise additional ingredients such as chloroquine, pro-tic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. Also comprised may be a surfactant, a wetting agent, a dispersing agent, a suspending agent, a buffer, a stabilizer or an isotonic agent. The above mentioned compounds are added in conditions respecting pH limitations.

The diagnostic composition of the present invention can also comprise a preservative. Preservatives according to certain compositions of the invention include, without limitation: bu-tylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

In a further embodiment the present invention relates to a kit for diagnosing, detecting, monitoring or prognosticating a pathologic status in a subject’s bone marrow. The kit may comprise one or more means for the determination of the presence of (i) CD71 and/or GPA, (ii) CD45, (iii) nucleic acids of rare circulating cells and at least one of (iv) or (v): (iv) EpCam and (v) vimentin, in a subject’s sample, e.g. as defined herein above. The kit may preferably comprise an antibody binding to CD71, and/or an antibody binding to GPA, and/or an antibody binding to CD45, and/or a dye which stains nucleic acids such as DAPI etc. The kit may, in yet another group of preferred embodiments additionally comprise means for the detection of the presence of one, two, three or all of CD44, CD24, CD133 and CD31, e.g. as defined herein above.

The kit may preferably be formulated as diagnostic composition, e.g. as has been described herein above and may comprise suitable carriers etc. The components or ingredients of the diagnostic kit may, according to the present invention, be comprised in one or more containers or separate entities. The nature of the agents is determined by the method of detection for which the kit is intended.

The kit may optionally comprise a package insert or a leaflet with instructions. The term “package insert” or “leaflet with instructions” is used to refer to instructions customarily included in commercial packages of diagnostic products that contain information about the usage, calibration and/or warnings concerning the use etc. The leaflet with instructions may be part of the kit.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Isolation, Dying and Analysis of Circulating Rare Cells

Exemplified is the preparation procedure of whole blood samples for the analysis of circulating rare cells denoted as cell-based liquid biopsy, herein employed to support fast fluorescence microscopic identification of erythroblasts, epithelial cells and those in epithelial to mesenchymal transition.

### Materials

10 ml whole blood from healthy adult donors stored for at most 24 hours in sodium heparin blood storage containers.
incubation buffer solution: iso-osmolaric phosphate buffered solution supplemented with 3% fetal bovine serum.
Washing solution: iso-osmolaric phosphate buffered solution
red blood cell lysis buffer: 154 mM NH4Cl, 10 mM NaHCO3, 2mM EDTA
hematology centrifuge equipped with swinging bucket rotator.
hemocytometer (Neubauer)
Antibody cocktail;
Permeabilization solution 2 for cell fixation (BD Bioscience)
Immuno-magnetic beads reactive against undesired cells (SanoLibio GmbH, Munich).
automated enrichment device (Walderbach Series, SanoLibio GmBH, Munich)
Perkin Elmar, Operetta high content imaging system.

### Experimental Procedure

Red blood cells were lysed as known to the expert in the field via suited lysis buffers (RBC lysis buffer). Purified white blood cells from initially 5 ml whole blood were concentrated in the separation buffer by centrifugation in a hematology centrifuge and rested for 15 minutes. The purified leukocyte stock concentration was assessed by common hemocytometer counting method (as required for optimal enrichment results) and then subjected to the automated enrichment procedure. The enriched sample was concentrated in 30 uL cell friendly solution and comprised blood circulating rare cells, leukocytes in range of 1000 to 3000 cells and cellular debris. Subsequent to enrichment, the cell suspension was subjected to fixation and perforation using the permeabilization solution 2 according to the manufacturers instructions and stained for fluorescence microscopy analysis using anti-CD45PE (ebioscience), anti-CD71FITC, anti-Ep-CamSB650 (ebioscience), a-VimentinFITC (ebioscience) and anti-pan-CKPerCPCy5 (ebioscience) each using 1 µL undiluted dye solution in the cold and dark for 25 minutes. Nucleus staining followed using 0.5 µL Hoechst 33342 DNA staining (ThermoFischer). The suspension was washed in 1.5 ml PBS and subsequently concentrated by entrifugation, 300xg 5 min at 4° C. The pellet was resuspended in 70 µL cell friendly solution and loaded into one well of a specialized 384-well plate suitable for high resolution image recording at 40x magnification using the Operetta system (PerkinElmar) recording a bright field channel, and channels for UV, green, yellow, orange and red fluorescence light emission. Columbus analysis software served as screening and image analysis tool. Marker positive cells included EpCam, CD71, cytokeratin and Vimentin and were identified by a cell-like round formation in case of membrane staining in congruency with bright field morphology, positive Hoechst staining in the absence of the typical ring formation as consequence of positive CD45PE staining throughout the emission light spectrum from 520 nm till 650 nm.

### Example 2

### Biomarker Type Classification by Morphology

### Materials

As described in Example 1.

### Experimental Procedure

The Assay procedure as described in Example 1 has been employed yielding cell material for morphological analysis.

Cell description:

TABLE 3

| Cell class | Name | Cell shape: size/roundness | Nucleus: size/roundness/chromatin condensation/NC-ratio |
|---|---|---|---|
| 1a | Mature EB | Oval to round/<11.5 um | 3 to 7 um/round highly to moderate condensed chromatin/high to moderate NC-ratio |
| 1b | Giant EB | Oval or round/>13 um | 6.5 to 9 um/round/low density chromatin/low N/C-ratio |
| 1c | Megaloblast | Shape variable/>9 um | >7.5 um/variable/intra nuclear variation from high to low/moderate to high NC-ratio |
| 1d | Macronormoblast | Round/>10 um | 3-5 um/round/high density chromatin/very low NC-ratio |
| 2a | Bi nucleated EB | Oval round/any size | Any size/round/any density/any NC-ratio |
| 2b | Multi nucelated EB | Oval round/any size | Any size/round/any density/any NC-ratio |
| 3a | Mitotic EB without nuclear bridge | Oval round/any size | Any size/round/any density/any NC-ratio |
| 3b | Mitotic EB with nulceic abnormality and/or nuclear bridge | Oval round/any size | Any size/round/any density/any NC-ratio |
| 4 | EB cluster with or without nuclear bridge | Oval round/any size | Any size/round/any density/any NC-ratio |

### Example 3

### Healthy donor tests

Healthy donor tests were conducted to assess cut-off values for abnormality with respect to given cells types listed in Table 4.

### Materials and general procedure:

As described in Example 1.

### Study procedure:

The abnormality (=specificity) cut-off was determined by taking into account limit of blank (LoB) and limit of detection (LoB). LoB is the highest apparent analyte concentration expected to be found when replicates of a blank sample containing no analyte are tested and followed the calculation:

LoB = meanblank + 1.645(SDblank);

The LoD is the lowest analyte concentration likely to be reliably distinguished from the LoB and at which detection is feasible. It utilises both the measured LoB and test replicates of a sample known to contain a low concentration of analyte and was calculated as follows:

LoD = LoB + 1.645(SD low concentration sample). The cut-off equals the limit of detection.

The cut-off values are deduced from measurements of 15 healthy donors as determined by subjective well being, absence of illness for the last 2 months, and a normal blood test including C-Reactive protein with values in normal range. LoB and LoD are given in cells per ml whole blood.

Variation in recovery of low concentrated cells was measured in triplicates spiking 10 tumor cells (MCF-7) in a 2.5x107 leukocyte suspension with subsequent identification as described in example 1. The variation was assumed valid for all cell types, therefore using the result all LOD calculations.

### Study findings:

See Table 4, infra

TABLE 4

| Cell type | Cell frequency | Limit of Blank | Limit of Detection | Abnormality cut-off |
|---|---|---|---|---|
| CTC - Ep-Cam+/CD45-Hoechst+ | 2 out 15 donors counted 1 cell per 2.5 ml blood) | 0.3 cells per ml | SDₛₚᵢₖₑ = 1 cell variation for 10 cells 1.9 cells per ml | 2 cells per ml |
| EB class 1a-Normal erythroblast | 15/15 donors counting 5 cells per ml on average | 14.9 cells per ml | 16.7 cells per ml | 17 cells per ml |
| EB class 1b giant erythroblast | 7/15 donors counting 0.43 cells per ml on average | 1 cells per ml | 2.6 cells per ml | 3 cells per ml |
| EB class 1c -Megaloblast | 1/15 donors counting 1 cell | 0.5 cells per ml | 2.1 cells per ml | 2 cells per ml |
| EB class 1d | 3/15 donors counting 1 cells | 0.7 cells per ml | 2.4 cells per ml | 2.5 cells per ml |
| All other EB classes | Not detected | 0 | 1.6 cells per ml | 1.6 cells per ml |

### Example 4

### Metastatic Breast Cancer Patient

Exemplified is the biomarker analysis with listed cell types (see table) for the identification of invasive breast cancer with bone metastasis.

### Materials and General Procedure

As described in Example 1.

### Study Procedure:

The ratio between normal and abnormal Ebs is a requirement to exclude benign bone marrow disorders with a cut-off of 50 was calculated as follows; the count of class 1a cells (see Table 4) per ml was divided by the sum of the counts of all classes 1c to 4 per ml.

The breast cancer patient was a formerly early stage breast cancer patient that showed recurrence after 5 years with malignancy in liver and bone. The patient was treatment-naive at the time of liquid biopsy.

### Study Findings

The patient’s malignancy has been confirmed by liquid biopsy, having detected circulating epithelial cells above abnormality cut-off. A quantitative impression of a marked bone marrow damage is given by the very low ratio number between normal and abnormal EBs, measuring 0.83. The EB profile indicates severe bone marrow damage and is suggestive of bone metastasis. The class 1b-d are common findings in bone marrow disorders, as such should be found in the blood of the herein investigated patient at higher levels. The cut-off value given in Table 6 stratifies between normality and abnormality only as such is the lowest possible cut-off for bone marrow disorder. The patient’s EB concentrations are markedly increased above abnormality cut-off. The clinical indication is related to the patient EB concentration.

TABLE 5

| CTC class | Nr. per ml | Abnormality cut-off (positive events per ml) | Clinical Indication |
|---|---|---|---|
| EpCam+/CD45-Hoechst positive | 10 | 2 | Malignancy |

TABLE 6

| | | | |
|---|---|---|---|
| EB class | Nr. per ml | Abnormality cut-off (positive events per ml) | Clinical indication |
| 1a | 486 | 17 | Mild Bone marrow disorder |
| 1b | 65 | 3 | General Bone marrow disorder |
| 1c | 24 | 2 | General bone marrow disorder |
| 1d | 28 | 2.5 | General bone marrow disorder |
| 2a | 66 | 1.6 | Severe damage - metastasis |
| 2b | 0.4 | 1.6 | Severe damage |
| 3a | 112 | 1.6 | Severe damage |
| 3b | 0 | 1.6 | No damage |
| 4 | 290 | 1.6 | Severe damage |
| | | normal/abnormal Ratio | 0.83 |
| | | Conclusion | Highest degree of damage/prediction of metastasis |

### Example 5

### Metastatic Small Cell Lung Cancer Patient

Exemplified is the biomarker analysis with listed cell types (see table) for the identification of bone marrow involvement in extensive small cell lung cancer (SCLC) without bone metastasis.

### Materials and General Procedure

As described in Example 4.

### Study Procedure

The SCLC patient, male aged 59 years was symptomatic with hemoptysis. A CT scan of the chest revealed a mass at the right upper lung of 6.3 cm in size, multiple mediastinal lymph nodes and a subcutaneous nodule in the mid abdomen, 4 cm in size. Brochoscopy and biopsy confirmed extensive stage small cell lung cancer. Liquid Biopsy blood test was conducted during 3rd line therapy before the 3 cycle having administered a cyclephosphamide, doxorubicin and vincristine regime, which was found chemoresponsive on the first cycle. The patient passed away 2 months later having developed multiple brain metastasis.

### Study Findings

In retrospective, malignancy was confirmed by cell-based liquid biopsy, detecting 47 circulating epithelial cells per ml whole blood. Systemic disease stage was supported by moderate grade bone marrow damage (see Table 6). The ratio between normal and abnormal EBs measured 6.22, being affirmative for bone marrow disorder and suggesting involvement of disseminated tumor cells. The EB profile including the relative low ratio number suggests a moderate bone marrow damage, which does not support a diagnosis of bone metastasis and complies with the clinical findings. The results may support the general notion, that SCLC produces much lesser bone marrow tumor cell dissemination that for example breast cancer cells (see Example 4).

TABLE 7

| CTC class | Nr. per ml | Abnormality cut-off | Clinical Indication |
|---|---|---|---|
| EpCam+/CD45-Hoechst positive | 47 | 2 | Malignancy |

TABLE 8

| | | | |
|---|---|---|---|
| EB class | Nr. per ml | Abnormality cut-off | Clinical indication |
| 1a | 77 | 17 | General Bone marrow disorder |
| 1b | 2 | 3 | Statistical insignificance |
| 1c | 0.8 | 2 | Statistical insignificance |
| 1d | 0 | 2.5 | No damage |
| 2a | 7.2 | 1.6 | moderate damage |
| 2b | 0 | 1.6 | No damage |
| 3a | 1.2 | 1.6 | Statistical insignificance |
| 3b | 0.4 | 1.6 | Statistical insignificance |
| 4 | 0.8 | 1.6 | Statistical insignificance |
| | | normal/abnormal Ratio | 6.22 |
| | | Conclusion | Moderate bone marrow damage |

### Example 6

### Healthy Donor with Underlying Chronic Conditions

Exemplified is the biomarker analysis with listed cell types (see table 3) for the identification of bone marrow involvement in a healthy donor with underlying confirmed stable thrombocytosis and another donor afflicted with diabetes.

### Materials and General Procedure

As described in Example 4.

### Study Procedure

Two healthy female donors, aged 60 years (D1) and 43 years (D2) with underlying thrombocytosis and diabetes type II, respectively were tested for the analysis of their EB profile thereby donating 10 ml blood. The first donor’s thrombocyte count has remained stable being asymptomatic over the past 6 years, measuring a tripled amount when compared to the healthy standard that measures roughly 3x10⁵ platelets per µL. The diabetic patient showed mild stable disease.

### Study Findings

No circulating epithelial cells were found in both donors, ruling out any malignancy. In donor 1, one class 3a cell has been identified in 2.5 mL blood. In support of non-malignancy, the ratio between normal and abnormal EBs measured high. However, a marked increase in EB cell type 1a suggests abnormality in the bone marrow. Low grade damage was concluded and supported by the finding of class 3a cells, yet below LOD. Donor 2 measured statistical insignificant erythroblast abnormality with presence of class 2a and 3a cells indicating mild bone marrow damage.

TABLE 9

| CTC class | Nr. per ml | Abnormality cut-off | Clinical Indication |
|---|---|---|---|
| EpCam+/CD45-Hoechst positive | D1: 0 D2: 0 | 2 | No indication |

TABLE 10

| | | | |
|---|---|---|---|
| EB class | Nr. per ml | Abnormality cut-off | Clinical indication |
| 1a | 541 | 17 | D1: Mild bone marrow damage D1: no damage |
| 1b | 0.3 | 3 | D1: Statistical insignificance D2: No damage |
| 1c | 0 | 2 | No damage |
| 1d | 0 | 2.5 | No damage |
| 2a | 0 | 1.6 | D1: No damage D2: Mild bone marrow damage/statistical insignificant |
| 2b | 0 | 1.6 | No damage |
| 3a | 0.3 | 1.6 | D1: Mild damage/Statistical insignificance D2: Mild damage/Statistical insignificance |
| 3b | 0 | 1.6 | No damage |
| 4 | 0 | 1.6 | No damage |
| | | Normality/Abnormality ratio | D1: 902 D2: 105 |
| | | Conclusion | Low grade bone marrow damage |

### Example 7

### Minimal Residual Disease in Early Stage Breast Cancer

Exemplified is the biomarker profile analysis with listed cell types (see table 3) for the identification of minimal residual disease in a stage II post surgery breast cancer patient.

### Materials and General Procedure

As described in Example 4.

### Study Procedure

The donor, female aged 37 years, diagnosed with stage II hormone low risk positive breast cancer was tested 4 months after tumor resection during adjuvant therapy for the analysis of her EB and CTC profile thereby donating 10 ml peripheral blood.

### Study Findings

A low number of circulating epithelial cells were found, ruling in residual malignancy in the donor. The ratio between normal and abnormal EBs was low. Therefore, low grade bone marrow damage has been assessed and is supported by the finding of class 2a and 3a cells, yet below LOD. The findings suggest that the possible origin of CTC can be attributed to bone marrow dissemination there, causing low grade bone marrow damage and concluding with low grade distant active minimal residual disease.

TABLE 11

| CTC class | Nr.per ml | Abnormality cut-off | Clinical Indication |
|---|---|---|---|
| EpCam+/CD45-Hoechst positive | 2.6 | 2 | Minor residual malignancy |

TABLE 12

| | | | |
|---|---|---|---|
| EB class | Nr. per ml | Abnormality cut-off | Clinical indication |
| 1a | 12.4 | 17 | Mild bone marrow damage |
| 1b | 0.6 | 3 | Statistical insignificance |
| 1c | 1.2 | 2 | Mild bone marrow damage |
| 1d | 0 | 2.5 | No damage |
| 2a | 0.2 | 1.6 | Mild bone marrow damage |
| 2b | 0 | 1.6 | No damage |
| 3a | 0.4 | 1.6 | Mild bone marrow damage |
| 3b | 0 | 1.6 | No damage |
| 4 | 0 | 1.6 | No damage |
| | | Normality/Abnormality ratio | 7.2 |
| | | Conclusion | Low grade bone marrow damage |

### Example 8

### Solid Tissue Cancer Staging

Exemplified is a possible translation of the said biomarkers into clinical cancer care as an auxiliary diagnostic test being complementary to staging histopathological lesions, in particular carcinomas, melanomas and sarcomas.

### Materials and General Procedure

As described in Example 1.

### Diagnostic Test Rationale and Procedure

The cellular blood test herein donated as fluid biopsy is carried out in parallel with tissue biopsy for histopathological investigations purposed to increase certainty about metastatic status of cancers. Conventional means of staging, for example in breast cancer diagnostics, target tumor tissue as well as nearby lymph nodes as a first impression of the tumor system. However, distant tumor cell dissemination in particular at very low amounts such as into the bone marrow is often underdiagnosed, leading to understaging. The fluid biopsy test is scoring bone marrow damage and thus contributes to conventional staging, potentially increasing accuracy in the detection of metastatic cancer for improved therapy decision making. In contrast to tissue biopsy or molecular based investigations, the fluid biopsy test does not rely on prediction of distant tissue invasion rather constituting a real-time assessment of the ongoing tumor evolution. Hereby, the fluid biopsy test may not necessarily rely on the confirmation of malignancy by histopathology yielding a diagnosis of malignancy on its own as well as yielding a result about bone marrow damage. Together with the diagnosis of malignancy by either method (fluid or tissue biopsy), bone marrow damage can be interpreted as result of DTC invasion.

According to the fluid biopsy test, bone marrow tumor cell infiltration hence, systemic spread, would be signalled by the presence of abnormal quantities and/or types of circulating erythroblasts. Consequently, the tests interprets bone marrow damage as “excluding distant invasion”, “invasion in suspicion” and “distant invasive cancer”. The latter case is indicated by severe and moderate bone marrow damage in the presence of EB aberration. “Invasion in suspicion” is indicated by moderate (only class 1ab) and mild damage (class 1cd) and “no invasion” can be diagnosed upon sub cut-off biomarker concentrations. The diagnosis of “invasive cancer” by the fluid biopsy strongly suggests metastatic disease and is independent of histopathological staging. On the other hand, invasion in suspicion or no invasion shall follow histopathological staging. As such, the fluid biopsy test does not reproduce all other aspects of conventional staging, hence the use as complementary or auxiliary test.

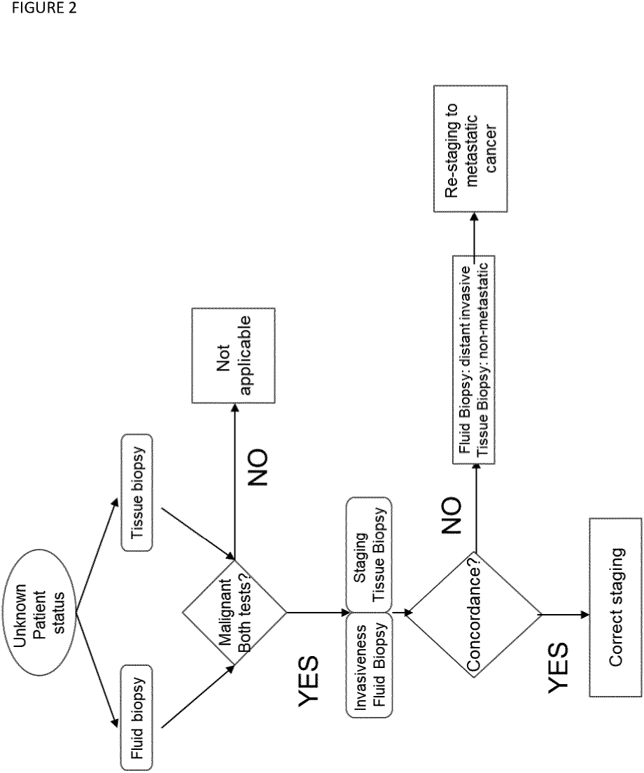

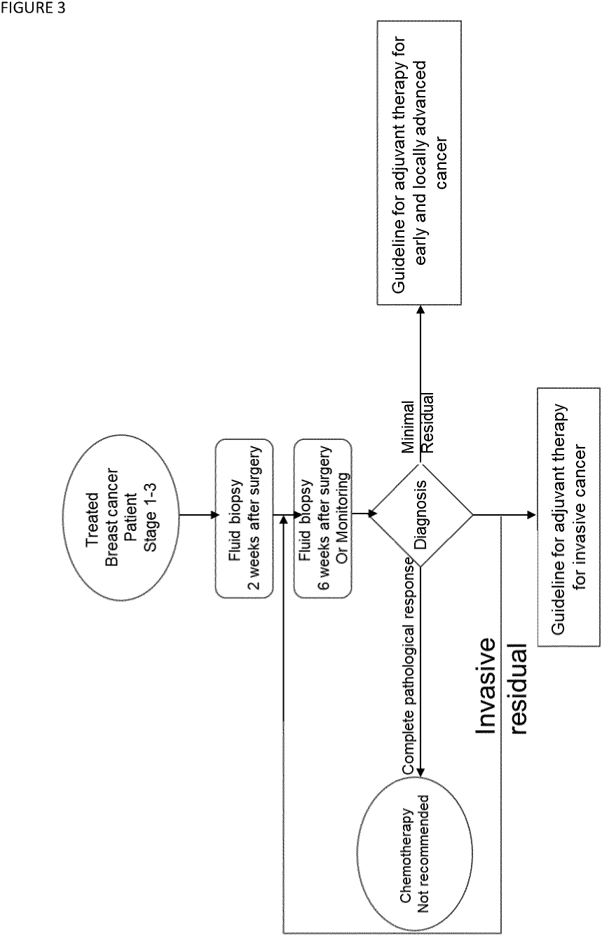

The diagnostic procedure is demonstrated in FIGS. 2 and 3. The procedure starts with the tissue as well as fluid biopsy in parallel on individuals in suspicion of cancer. The diagnosis of malignancy in a treatment naive individual must be in concordance between the two as to proceed. In fluid biopsy, malignancy is revealed yet not ascertained by the presence of CTCs greater a given cut-off (see Example 3). If in concordance with tissue biopsy, the state of invasiveness shall be compared. The testing procedure provides patient benefit in case of positive distant invasive cancer detection by fluid biopsy and negative distant invasion by conventional means. Consequently, re-staging is advised potentially requiring stage 4 treatment. In any other case, such as “no invasion” or “invasion under suspicion” by fluid biopsy, results have no relevance to conventional staging relying solely on histopathology prediction and/or specialized diagnostics to reveal metastasis.

1. A method for detecting, diagnosing, monitoring or prognosticating a pathologic status in a subject’s bone marrow comprising at least the step of determining the presence of aberrant erythroblasts in a subject’s bodily fluid sample.

2. The method of claim 1, wherein said sample is a venous blood sample or a peripheral blood sample, preferably an inferior vena cava sample or a portal vein sample.

3. The method of claim 1 or 2, wherein said determination comprises determining the ploidy of said erythroblasts and the potential presence of (i) a nuclear budding or lobulation phenotype, (ii) an internuclear bridge, (iii) two or more nuclei in a single cell, (iv) megaloblasts, (v) macronormoblasts, (vi) erythroblasts in synchronous cytoplasmic division, (vii) erythroblast aggregates comprising at least 3 adherent erythroblasts, (viii) increased ploidy within the erythroblasts’ nuclei.

4. The method of claim 3, wherein said aberrant erythroblast shows an increased ploidy, preferably has a bi- or multi-nuclear phenotype.

5. The method of claim 3, wherein said determination additionally comprises ascertaining the presence of at least CD71 and/or GPA, and optionally one or more of CD44, CD45, VAV1, Kell blood group protein and of nucleic acids in cells of the subject’s sample.

6. The method of claim 5, wherein the identification of aberrant erythroblasts showing an increased intranuclear and/or intracellular ploidy, a nuclear budding or lobulation phenotype, an internuclear bridge, two or more nuclei in a single cell, a megaloblast appearance, a macronormoblast appearance and simultaneously the identification of a CD71⁺ (positive) and/or GPA⁺(positive); and CD45⁻ (negative) biochemical status in said erythroblasts in the sample is indicative for bone marrow disorders such as myelodysplastic syndrome/acute myeloid leukemia, lymphomas, essential thrombocythemia or diabetes mellitus.

7. The method of claim 5, wherein said determination additionally comprises ascertaining the presence of EpCam and cytokeratin and optionally of vimentin, in cells of the subject’s sample.

8. The method of claim 5 or 7, wherein said determination additionally comprises ascertaining whether cells the subject’s sample are present in the form of cell clusters.

9. The method of claim 7 or 8, wherein the identification of aberrant erythroblasts showing an increased intranuclear and intracellular ploidy, a nuclear budding or lobulation phenotype, an internuclear bridge, two or more nuclei in a single cell, a megaloblast appearance, a macronormoblast appearance and simultaneously the identification of a CD71⁺ (positive) and/or GPA⁺ (positive) and CD45⁻ (negative) biochemical status in said erythroblasts in the sample, as well as the identification of EpCam⁺ (positive) or cytokeratin⁺ (positive) cells in the sample is indicative for an invasive solid tissue cancer.

10. The method of claim 7 or 8, wherein said determination additionally comprises a morphological analysis of cells in said sample, preferably after May-Grunwald-Giemsa (MGG) staining of the cells.

11. The method of claim 10, wherein said morphological analysis of cells comprises a classification of the cells in the sample into:
class 1, wherein the sample comprises cells which are round or oval and which comprise one nucleus;
class 2, wherein the sample comprises cells which are round or oval and which comprise at least two nuclei;
class 3, wherein the sample comprises pairs of cells comprising a constriction; and
class 4, wherein the sample comprises aggregations of at least three round or oval cells with one or more nucleus or nuclei.

12. The method of claim 11, wherein said class 1 comprises a further sub-classification of the cells into:
class 1a, wherein the sample comprises normal erythroblasts with a diameter from about 6.5 µm to 12.4 µm with dense nucleus and high nucleus to cytoplasm ratio;
class 1b, wherein the sample comprises giant circulating erythroblasts of a diameter of >12.5 µm, of at least one low density nucleus in a diameter of about 6 to 10 µm and low nucleus to cytoplasm ratio
class 1c, wherein the sample comprises megaloblasts with nucleocytoplasmic asynchrony and moderate to high density chromatin and high nucleus to cytoplasm ratio; and
class 1d, wherein the sample comprises macronormoblasts with no nucleocytoplasmic asynchrony, total condensation nuclei in a diameter of about 4 µm to 6 µm and a low nucleus to cytoplasm ratio.

13. The method of claim 11, wherein said class 2 comprises a further sub-classification of the cells into:
class 2a, wherein the cells are bi-nucleated; and
class 2b, wherein the cells contain at least 3 nuclei.

14. The method of claim 11, wherein said class 3 comprises a further sub-classification of the cells into:
class 3a, wherein the cell shows no nuclear bridge; and
class 3b, wherein the cell shows a nuclear bridge and wherein the nuclei show an inequality in size, shape and/or chromatin density.

15. The method of any one of claims 10 to 14, wherein said determination additionally comprises the determination of the ratio of at least one of: (i) class 1a cells vs. class 1c/1d cells; (ii) class 1a cells vs. class 2 cells; and (iii) class 1a cells vs. class 3 cells, and of clusters of circulating tumor cells (CTCs).

16. The method of claim 15, wherein the identification of at least one of (i) to (iii):
(i) an EpCam⁺ (positive) and CD45⁻ (negative) circulating tumor cell (CTC) in the sample (marker 1);
(ii) a CTC cell cluster with a single cell diameter of 6 to 20 µm of CTCs showing marker 1 in the sample (marker 2);
(iii) an EpCam⁻ (negative) and vimentin⁺ (positive) CTC in the sample (marker 3); and additionally of
(iv) the presence of a ratio of at least one of (i) class 1a cells vs. class 1c/1d cells; (ii) class 1a cells vs. class 2 cells; and (iii) class 1a cells vs. class 3 cells of 50 or less to 0 is indicative for the malignancy of a solid tissue cancer.

17. The method of claim 12, wherein the identification of:
class 1a cells present in an amount of about 10 to 500 cells per ml in the sample and of class 1b/1c/1d cells present in an amount of about 1 to 10 cells per ml in the sample is indicative for a mild bone marrow damage; or
class 1a cells present in an amount about 1000 to 5000 cells per ml in the sample and of class 1b/1c/1d cells present in an amount of about 10 to 50 cells per ml in the sample is indicative for a moderate bone marrow damage; or
class 1a cells present in an amount greater than about 10 000 cells per ml in the sample and of class 1b/1c/1d cells present in an amount greater than about 10 cells per ml in the sample is indicative for a severe bone marrow damage.

18. The method of claim 13, wherein the identification of class 2a and class 2b cells in an amount of about 0.5 to 10 cells per ml of sample, is indicative for an moderate bone marrow damage; or wherein the identification of class 2a cells and class 2b cells in an in an amount of about 10 to 50 cells per ml of sample, is indicative for a severe bone marrow damage.

19. The method of claim 14 wherein the identification of class 3a cells in an amount of about 1 to 10 cells per ml of sample, is indicative for a moderate bone marrow damage; or wherein the identification of class 3a cells in an amount of about 10 to 50 cells per ml of sample and/or class 3b cells in an amount of about 1 to 10 cells per ml of sample is indicative for a severe bone marrow damage.

20. The method of claim 11, wherein the identification of class 4 cells in an amount of a least one cell per ml of sample is indicative for a severe bone marrow damage.

21. The method of claim 17, wherein the identification of a mild or moderate bone marrow damage and the identification of marker 1, 2 and/or 3 as defined in claim 16 (i) to (iii) and the identification of a ratio of 50 or less to 0 as defined in claim 16 (iv) is indicative for a cancer treatment related bone marrow damage of non-invasive cancer or a dormant cancer disease, minimal residual cancer disease or micro-metastasis in the bone marrow related to invasive solid tissue cancer.

22. The method of claim 18, or 19, wherein the identification of a moderate bone marrow damage in the absence of class 2b cells per ml of sample and the identification of marker 1, 2 and/or 3 as defined in claim 16 (i) to (iii) and the identification of a ratio of 50 or less to 0 as defined in claim 16 (iv) is indicative for the presence of a progressive micro-metastasis in the bone marrow related to an invasive solid tissue cancer.

23. The method of claim 17, wherein the identification of a severe bone marrow damage and the identification of marker 1, 2 and/or 3 as defined in claim 16 (i) to (iii) and the identification of a ratio of 50 or less to 0 as defined in claim 16 (iv) is indicative for the presence of an active micro-metastasis in the bone marrow related to an invasive solid tissue cancer.

24. The method of claim 18, 19 or 20, wherein the identification of a severe bone marrow damage in the absence of class 2b cells per ml of sample and the identification of marker 1, 2 and/or 3 as defined in claim 16 (i) to (iii) and the identification of a ratio of 50 or less to 0 as defined in claim 16 (iv) is indicative for a bone metastasis related to invasive solid tissue cancer or primary bone cancer.

25. Use of aberrant erythroblasts as a marker for diagnosing, detecting, monitoring or prognosticating a pathologic status in a subject’s bone marrow.

26. The use of claim 25, wherein said aberrant erythroblasts are present in amounts as defined in any one of claims 17 to 20.

27. The use of claim 26, wherein said aberrant erythroblasts are used in combination with additional markers as defined in claim 16.

28. The use of claim 27, wherein said pathologic status is malignant solid tissue cancer, preferably non-invasive solid tissue cancer in the presence of mild or no bone marrow damage.

29. A composition for diagnosing, detecting, monitoring or prognosticating a pathologic status in a subject’s bone marrow, comprising means for the determination of the presence of (i) CD71 and/or GPA, (ii) CD45, (iii) nucleic acids of rare circulating cells and at least one of (iv) or (v): (iv) EpCam and (v) vimentin, in a subject’s sample.

30. The composition of claim 29, wherein the means for the detection of EpCam are anti-EpCam antibodies MH99 and/or VU-1D9.

31. The composition of claim 29 or 30, additionally comprising means for the detection of the presence of one, two, three or all of CD44, CD24, CD133 and CD31.
